(19) United States
(12) Patent Application Publication
(10) Pub. No.: US 2026/0290492 A1
(43) Pub. Date: Sep. 24, 2026

(54) METHODS FOR IDENTIFYING BIOLOGICAL CHARACTERISTICS OF A COMPLEX MOLECULAR STRUCTURE AND RELATED COMPUTER PROGRAM

(71) Applicants: CENTRE NATIONAL DE LA RECHERCHE SCIENTIFIQUE, Paris (FR); UNIVERSITE PARIS-SACLAY, Gif-sur-Yvette (FR); UNIVERSITY OF HOUSTON, Houston, TX (US); ECOLE POLYTECHNIQUE, Palaiseau (FR)

(72) Inventors: George E. FOX, Houston, TX (US); Madhan TIRUMALAI, Houston, TX (US); Quyen D. TRAN, Beaumont, TX (US); Laurie CALVET, Versailles (FR)

(21) Appl. No.: 19/484,551

(22) PCT Filed: May 16, 2024
(86) PCT No.: PCT/EP2024/063605

§ 371 Date: Nov. 14, 2025

Related U.S. Application Data
(60) Provisional application No. 63/466,960, filed on May 16, 2023.

(51) Int. Cl. G16B 15/00; G16B 5/00

(52) U.S. Cl. CPC G16B 15/00; G16B 5/00

(57) ABSTRACT

The present inventions concerns a method for identifying at least one biological characteristic of a complex molecular structure, the method comprising producing an interaction network representation of the complex molecular structure from a structural file, wherein said representation comprises nodes connected by edges, and using the interaction network representation to identify said at least one biological characteristic of the complex molecular structure. The present inventions also concerns a computer program comprising software instructions which, when executed by a computer, implement a method according to the invention.

### CROSS-REFERENCE TO RELATED APPLICATION

This application claims the benefit of and priority to the U.S. Provisional Application Ser. No. 63/466,960, titled “METHODS FOR DEVELOPMENT OF THERAPEUTICS BASED ON MODELING OF RIBOSOME STRUCTURES AND INTERACTIONS,” filed on May 16, 2023, the disclosure of which is incorporated herein by reference in their entirety.

### FIELD

Aspects of this disclosure provide a method for identifying at least one biological characteristic of a complex molecular structure, the method comprising producing an interaction network representation of the complex molecular structure from a structural file, wherein said representation comprises nodes connected by edges, and using the interaction network representation to identify said at least one biological characteristic of the complex molecular structure. The present inventions also concerns a computer program comprising software instructions which, when executed by a computer, implement a method according to the invention.

### BACKGROUND

Among complex molecular structures, nucleoproteins comprise proteins and nucleic acid (such as DNA or RNA). Nucleoproteins include for examples ribosomes, nucleosomes and viral nucleocapsids. The ribosome is a complex dynamic nano-scale machine that reads a gene template from a messenger RNA (mRNA) and translates it into a chain of amino acids, synthesizing a protein. Over a span of twenty years or more, scientists have produced and refined near-atomic resolution crystal structures via x-ray diffraction and cryo-electron microscopy images, which are snapshots of its dynamics in action and have enabled greater understanding of its function, but a concise representation facilitating comparisons and identifying changes in global structure is lacking.

### SUMMARY

The inventors have succeeded in representing the ribosome as a coarse grain network of elements (nodes) and their connections (interactions or edges), thereby allowing for its concise description (see FIG. 2). Thereby the inventors have found a new method for identifying at least one biological characteristic of a complex molecular structure comprising producing an interaction network representation of it. Indeed this representation enables systematic comparisons and new analysis of complex molecular structures revealing new biological characteristics.

The invention relates to a method, preferably a computer implemented method, for identifying at least one biological characteristic of a complex molecular structure comprising:

S1) producing an interaction network representation of the complex molecular structure from a structural file of said complex molecular structure, wherein said representation comprises nodes connected by edges, each node representing either a molecule or a part of a molecule of the complex molecular structure, according to the following:
i. determining each functional structure of each molecule of the complex molecular structure as a respective node,
ii. for each node, calculating the node isolated solvent surface accessible area from the structural file using a pseudo probe solvent molecule,
iii. for each possible pair of nodes, calculating the pair's isolated solvent surface accessible area from the structural file, using a pseudo probe solvent molecule,
iv. when the pair's isolated solvent surface accessible area is inferior to the sum of the isolated solvent surface accessible area of each node taken alone, then representing an edge connecting the two nodes of the pair,

S2) using the interaction network representation to identify at least one biological characteristic of the complex molecular structure.

Preferably in step ii) and iii), the b-factor of said node(s) in said structural file is taken into account to calculate the radius of the pseudo probe solvent molecule (also named pseudo probe).

This method for identifying at least one biological characteristic of the complex molecular structure is typically carried out by an electronic identification system.

The electronic identification system is configured for identifying at least one biological characteristic of the complex molecular structure and comprises a production module and a use module.

The production module is configured for producing an interaction network representation of the complex molecular structure from a structural file of said complex molecular structure, wherein said representation comprises nodes connected by edges, each node representing either a molecule or a part of a molecule of the complex molecular structure, according to the following:
i. determining each functional structure of each molecule of the complex molecular structure as a respective node,
ii. for each node, calculating the node isolated solvent surface accessible area from the structural file using a pseudo probe solvent molecule,
iii. for each possible pair of nodes, calculating the pair's isolated solvent surface accessible area from the structural file, using a pseudo probe solvent molecule,
when the pair's isolated solvent surface accessible area is inferior to the sum of the isolated solvent surface accessible area of each node taken alone, then representing an edge connecting the two nodes of the pair.

The use module is configured for using the interaction network representation to identify at least one biological characteristic of the complex molecular structure.

For example, the electronic identification system comprises an information processing unit consisting, for example, of a memory and a processor associated with the memory.

In this example, the production module and the use module are each implemented in the form of software, or a software brick, executable by the processor. The memory of the electronic identification system is then able to store production software and use software. The processor is then able to execute each of the production software and the use software.

In a variant not shown, the production module and the use module are each implemented as a programmable logic component, such as an FPGA (Field Programmable Gate Array), or as a dedicated integrated circuit, such as an ASIC (Application Specific Integrated Circuit).

When the electronic identification system is implemented in the form of one or more software programs, i.e. in the form of a computer program, it can also be recorded on a computer-readable medium (not shown). The computer-readable medium is, for example, a medium capable of storing electronic instructions and of being coupled to a bus of a computer system. By way of example, the readable medium is an optical disk, a magneto-optical disk, a ROM memory, a RAM memory, any type of non-volatile memory (e.g. EPROM, EEPROM, FLASH, NVRAM), a magnetic card or an optical card. A computer program containing software instructions is stored on the readable medium.

According to an embodiment, the step S2 comprises applying a mathematical transformation to the interaction network representation obtained in S1 to obtain a matrix, the matrix including rows of length N, where N is the total number of elements in the interaction network representation, each row representing a single element in the set of elements of the interaction network representation.

Preferably, each column represents a respective property of the interaction network representation.

According to an embodiment of the method, the identified biological characteristic of the complex molecular structure is a therapeutical target on said complex molecular structure, and wherein step S2) comprises:
a) finding paths connecting predefined regions of interest of the complex molecular structure and comprising less than 5 nodes, and
b) identifying as a therapeutical targets nodes from the paths found in step a), wherein paths are constituted of connected nodes and edges, each path comprising at least two nodes.

According to another embodiment of the method, said method comprises:
a) producing several interaction network representations of a complex molecular structure in different configurations according to step S1),
b) training an autoencoder, wherein said autoencoder comprises an encoder, a decoder connected at output of the encoder, wherein the output of the encoder is a latent space, and wherein the output of the decoder is similar to the input of the encoder, wherein interaction network representations of step a) are used as a training dataset of said autoencoder, and preferably
c) producing, as output of the trained autoencoder, simulated interaction network representations of said complex molecular structure, different simulated interaction network representations being produced by adapting respective parameters in the latent space.

Preferably, the latent space follows a distribution among a probability distribution and a normal distribution with mean and standard deviation as parameters of the latent space.

According to an embodiment of this method, the identified biological characteristic of the complex molecular structure is the region targeted by an active agent on said complex molecular structure, said method comprising:
a) producing several interaction network representations of a complex molecular structure in different configurations and simulated interaction network representations of said complex molecular structure,
b) producing an interaction network representation of said complex molecular structure with the active agent according to step S1),
c) determining a similarity between the interaction network representation of step b) and each interaction network representations of step a), from their output in the latent space, and selecting the network representation of step a) the most similar to the network representation of step b),
d) identifying the region of the complex molecular structure wherein paths have changed between the network representation of step c) and the network representation of step b), as the region of the complex molecular structure targeted by the active agent.

According to another embodiment of this method, the identified biological characteristic of the complex molecular structure is the activity of an agent on said complex molecular structure, the method comprising:
a. producing several interaction network representations of a complex molecular structure in different configurations and simulated interaction network representations of said complex molecular structure,
b. producing an interaction network representation of said complex molecular structure with the agent according to step S1),
c. determining the similarity between the interaction network representation of step b) and each interaction network representations of step a), from their output in the latent space, and selecting the network representation of step a) the most similar to the network representation of step b),
d. identifying the agent as active on the complex molecular structure when at least one of the following conditions is verified between the interaction network representation of step b) and the one selected in step c):
   i. the number of edges is significantly changed,
   ii. a path connecting predefined regions of interest of the complex molecular structure is changed,
wherein each path is constituted of connected nodes and edges, each path comprising at least two nodes,
   iii. the most similar network representation determined in step c) is a functional configuration of the complex molecular structure, and the similarity score between the interaction network representations of step c) and the interaction network representations of step b) is significantly low,
   iv. the most similar network representation determined in step c) is a non-functional configuration of the complex molecular structure.

A significantly changed number of edges means that the change in the number of edges is statistically significant compared to the relative metric or the greater than that expected from the algorithm that generated the interaction network representation. For instance for an autoencoder, the changes in the number of edges would represent a reconstruction error that is larger than the reconstruction error for a molecular structure without an antibiotic, for example at least 1% larger, preferably at least 5% larger, still preferably at least 10% larger.

For example, the number of edges is significantly changed when the number of edges varies by at least 1%, preferably by at least 5%, still preferably by at least 10%.

A significantly low similarity score means low enough so that the scores is not statistically significant compared to a ribosome known to be part of that representation but not used to train the method.

For example, the similarity score is significantly low when the similarity score is less than 0.2 or 20%, preferably less than 0.1 or 10%, still preferably less than 0.05 or 5%.

According to an embodiment of the method, the identified biological characteristic is the similarity of two configurations of a complex molecular structure, and said method comprises:
a) producing several interaction network representations of said complex molecular structure in different configurations according to step S1,
b) training an autoencoder, wherein said autoencoder comprises an encoder, a decoder connected at output of the encoder, wherein the output of the encoder is a latent space, and wherein the output of the decoder is similar to the input of the encoder, wherein interaction network representations of step a) are used as a training dataset of said autoencoder,
c) using as input to the autoencoder interaction network representations of the two configurations to compare, d determining a similarity between the two configurations from their output in the latent space and/or from their output of the decoder.

According to an embodiment, the similarity is evaluated from a distance between two interaction network representations from their output in the latent space and/or from a distance between two interaction network representations from their output of the decoder.

According to an embodiment of this method, the method allows identifying the specificity of an agent toward complex molecular structures, said method comprising:
a) realizing the method of the invention to identify the activity of the agent on a first complex molecular structure,
b) realizing the method of the invention to identify the activity of the same agent on a second complex molecular structure, and
c) when the agent is active on only one complex molecular structure, concluding that said agent is specific to said complex molecular structure.

The invention also relates to a computer program comprising software instructions which, when executed by a computer, implement a method of the invention.

Another aspect of this invention includes a computer-implemented method for calculating a sample large complex biomolecule based on a machine-learning (ML) model to validate whether a candidate drug compound is effective on the sample large complex biomolecule. The method includes identifying a target set of compounds based on one or more of: a defined target clinical application, a set of desired characteristics, or a defined class of compounds; pre-processing each compound of the target set of compounds to generate respective sets of feature data. The sets of feature data include a set of parameters based on a network analysis for the ML model to predict interaction of elements of the sample large complex biomolecule. The method further includes processing the sets of feature data with one or more trained machine learning models to produce predicted characteristic values for each compound of the target set of compounds for each of the set of desired characteristics. The one or more trained machine learning models are selected from a database of trained machine learning models based on at least the set of desired characteristics. The method further includes identifying a subset of the target set of compounds based on the predicted characteristic values.

According to an embodiment, the sample large complex biomolecule includes at least one of a ribosome or ribozyme including RNA and proteins decomposable into at least one of secondary structures, tertiary structures, or domains defined in a use case. The target set of compounds includes at least one of antibiotics, anti-viral drugs, or anti-cancer drugs. The sample large complex biomolecule is of bacterial or eukaryote origin.

According to an embodiment, the ML model includes a graphical neural network trained by operations including: receiving a training dataset of ribosomal structures; identifying one or more single elements for each of the training dataset of ribosomal structures; calculating, in the network analysis, interactions of the one or more single elements using a difference between (1) a sum of surface accessible solvent areas (SASAs) of the one or more single elements independently and (2) an actual surface accessible solvent area (SASA) of the one or more single elements together; and recording a set of parameters based on the network analysis for the ML model to predict the interaction of the elements of the sample large complex biomolecule.

According to an embodiment, the set of parameters includes at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph; a cluster coefficient of the network graph; a density of the network graph; an assortativity of the network graph; a neighborhood of one of the number of nodes. The one of the number of nodes has a maximum degree exceeding a threshold or a reference value; paths between two or more of the number of nodes having a respective maximum degree exceeding the threshold or the reference value; a number of residues associated with an output of the ML model, the output characterizing the interaction of elements of the sample large complex biomolecule.

According to an embodiment, the SASAs and SASA are computed using the Shrake Rupley method. Preferably, the radius of the solvent in said method should incorporate the b-factor of each node, using the pseudo solvent method described herein.

Another aspect of this invention includes a method for calculating a sample large complex biomolecule. The method includes identifying an action of a target set of compounds based on one or more of: a defined target clinical application, a set of desired characteristics, or a defined class of compounds; pre-processing each compound of the target set of compounds to generate respective sets of feature data wherein the sets of feature data include a set of parameters based on a network analysis for a machine learning (ML) model to predict interaction of elements of the sample large complex biomolecule; processing the sets of feature data with one or more trained machine learning models to produce predicted characteristic action for each compound of the target set of compounds for each of the set of desired characteristics. The one or more trained machine learning models are selected from a database of trained machine learning models based on at least the set of desired characteristics. The method further includes identifying a subset of the target set of compounds based on the predicted characteristic action.

According to an embodiment, the sample large complex biomolecule includes at least one of a ribosome or ribozyme including RNA and proteins decomposable into at least one of secondary structures, tertiary structures, or domains defined in a use case. The target set of compounds includes at least one of antibiotics, anti-viral drugs, or anti-cancer drugs. The sample large complex biomolecule is of bacterial or eukaryote origin.

According to an embodiment, the ML model includes a graphical neural network. The method further includes training the ML model by: receiving a training dataset of ribosomal structures; identifying one or more single elements for each of the training dataset of ribosomal structures; calculating, in the network analysis, interactions of the one or more single elements using a difference between (1) a sum of surface accessible solvent areas (SASAs) of the one or more single elements independently and (2) an actual surface accessible solvent area (SASA) of the one or more single elements together; and recording a set of parameters based on the network analysis for the ML model to predict the interaction of the elements of the sample large complex biomolecule.

According to an embodiment, the set of parameters includes at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph;
a cluster coefficient of the network graph; a density of the network graph; or an assortativity of the network graph; a neighborhood of one of the number of nodes. The one of the number of nodes has a maximum degree exceeding a threshold or a reference value; paths between two or more of the number of nodes having a respective maximum degree exceeding the threshold or the reference value; a number of residues associated with an output of the ML model, the output characterizing the interaction of elements of the sample large complex biomolecule.

According to an embodiment, the SASAs and SASA are computed using Shrake Rupley method. Preferably, the radius of the solvent in said method should incorporate the b-factor of each node, using the pseudo solvent method described herein.

Another aspect of this invention includes a method for a computer system to calculate a sample ribosome structure based on a machine-learning (ML) model, the method including: receiving an input of the sample ribosome structure for the ML model; processing the input to generate an output that characterizes an interaction of elements of the sample ribosome structure; and presenting, via a user-interface of the computer system, the output for validation or local targeting based on the interaction of the elements of the sample ribosome structure. The ML model has been trained using a training dataset of ribosomal structures by: receiving the training dataset of ribosomal structures; identifying one or more single elements for each of the training dataset of ribosomal structures; calculating, in a network analysis, interactions of the one or more single elements using a difference between (1) a sum of surface accessible solvent areas (SASAs) of the one or more single elements independently and (2) an actual surface accessible solvent area (SASA) of the one or more single elements together; and recording a set of parameters based on the network analysis for the ML model to predict the interaction of the elements of the sample ribosome structure.

According to an embodiment, identifying the one or more single elements for each of the training dataset of ribosomal structures includes: separating each of the training dataset of ribosomal structures into constituent elements, including at least one of: 5S rRNAs, tRNAs, mRNAs, ribosomal proteins, 16S rRNAs, or 23S rRNAs; and representing the constituent elements by a geometry having one or more perimeters.

According to an embodiment, the SASAs and SASA are computed using Shrake Rupley method. Preferably, the radius of the solvent in said method should incorporate the b-factor of each node, using the pseudo solvent method described herein.

According to an embodiment, the method further includes comparing, in the network analysis, the training dataset of ribosomal structures using either an unsupervised clustering algorithm including at least one of: K-means clustering, principle component analysis, or a latent space of an autoencoder or a supervised method, such as a multilayer perceptron, a convolutional neural network, a transformer, or a recurrent neural network.

According to an embodiment, the ML model includes a graphical neural network and wherein the set of parameters based on the network analysis for the ML model includes at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph; a cluster coefficient of the network graph; a density of the network graph; or an assortativity of the network graph; a neighborhood of one of the number of nodes. The one of the number of nodes has a maximum degree exceeding a threshold or a reference value; paths between two or more of the number of nodes having a respective maximum degree exceeding the threshold or the reference value; a number of residues associated with an output of the ML model, the output characterizing the interaction of elements of the sample large complex biomolecule.

According to an embodiment, the output includes at least one of: a plurality of paths connecting elements of interests of the sample ribosome structure; nodes that appear the most often in the plurality of paths; residuals associated with the output characterizing the interaction of elements of the sample ribosome structure for local targeting; or a subset of the plurality of paths susceptible to therapeutic targeting.

According to an embodiment, the presenting the output includes modeling an agent as one or more nodes of the network graph; and determining the agent as active when at least one of the following conditions is satisfied: a number of edges has been changed exceeding a threshold; a path connecting predefined regions of interest of the ribosome has changed; or a difference between the sample ribosome structure and a reference ribosome structure exceeds a threshold value.

Another aspect of this invention includes a system for calculating a sample ribosome structure based on a machine-learning (ML) model. The system includes: a user-interface; a memory; and a processing device coupled to the memory. The processing device and the memory are configured to: receive an input of the sample ribosome structure for the ML model; process the input to generate an output that characterizes an interaction of elements of the sample ribosome structure; and present, via the user-interface, the output for validation or local targeting based on the interaction of the elements of the sample ribosome structure. The ML model has been trained using a training dataset of ribosomal structures by: receiving the training dataset of ribosomal structures; identifying one or more single elements for each of the training dataset of ribosomal structures; calculating, in a network analysis, interactions of the one or more single elements using a difference between (1) a sum of surface accessible solvent areas (SASAs) of the one or more single elements independently and (2) an actual surface accessible solvent area (SASA) of the one or more single elements together; and recording a set of parameters based on the network analysis for the ML model to predict the interaction of the elements of the sample ribosome structure.

According to an embodiment, the processing device and the memory are configured to identify the one or more single elements for each of the training dataset of ribosomal structures by: separating each of the training dataset of ribosomal structures into constituent elements, including at least one of: 5S rRNAs, tRNAs, mRNAs, ribosomal proteins, 16S rRNAs, or 23S rRNAs; and representing the constituent elements by a geometry having one or more perimeters.

According to an embodiment, the SASAs and SASA are computed using the Shrake Rupley method. Preferably, the radius of the solvent in said method should incorporate the b-factor of each node, using the pseudo solvent method described herein.

According to an embodiment, the processing device and the memory are further configured to compare, in the network analysis, the training dataset of ribosomal structures using either an unsupervised clustering algorithm including at least one of: K-means clustering, principle component analysis, or a latent space of an autoencoder; or a supervised method such as a multilayer perceptron, a convolutional neural network, a transformer, or a recurrent neural network.

According to an embodiment, the ML model includes a graphical neural network. The set of parameters based on the network analysis for the ML model includes at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph; a cluster coefficient of the network graph; a density of the network graph; or an assortativity of the network graph; a neighborhood of one of the number of nodes. The one of the number of nodes has a maximum degree exceeding a threshold or a reference value; paths between two or more of the number of nodes having a respective maximum degree exceeding the threshold or the reference value; a number of residues associated with an output of the ML model, the output characterizing the interaction of elements of the sample large complex biomolecule.

According to an embodiment, the output includes at least one of: a plurality of paths connecting elements of interests of the sample ribosome structure; nodes that appear the most often in the plurality of paths; residuals associated with the output characterizing the interaction of elements of the sample ribosome structure for local targeting; or a subset of the plurality of paths susceptible to therapeutic targeting.

According to an embodiment, the presenting the output includes: modeling an agent as one or more nodes of the network graph; and determining the agent as active when at least one of the following conditions is satisfied: a number of edges has been changed exceeding a threshold; a path connecting predefined regions of interest of the ribosome has changed; or a difference between the sample ribosome structure and a reference ribosome structure exceeds a threshold value.

Another aspect of this invention includes a non-transitory computer-readable storage medium including instructions that, when executed by a processing device to calculate a sample ribosome structure based on a machine-learning (ML) model, cause the processing device to: receive an input of the sample ribosome structure for the ML model; process the input to generate an output that characterizes an interaction of elements of the sample ribosome structure; and present, via a user-interface, the output for validation or local targeting based on the interaction of the elements of the sample ribosome structure. The ML model has been trained using a training dataset of ribosomal structures by: receiving the training dataset of ribosomal structures; identifying one or more single elements for each of the training dataset of ribosomal structures; calculating, in a network analysis, interactions of the one or more single elements using a difference between (1) a sum of surface accessible solvent areas (SASAs) of the one or more single elements independently and (2) an actual surface accessible solvent area (SASA) of the one or more single elements together; and recording a set of parameters based on the network analysis for the ML model to predict the interaction of the elements of the sample ribosome structure.

According to an embodiment, the processing device is further to identify the one or more single elements for each of the training dataset of ribosomal structures by: separating each of the training dataset of ribosomal structures into constituent elements, including at least one of: 5S rRNAs, tRNAs, mRNAs, ribosomal proteins, 16S rRNAs, or 23S rRNAs; and representing the constituent elements by a geometry having one or more perimeters.

According to an embodiment, the processing device is further to compare, in the network analysis, the training dataset of ribosomal structures using either an unsupervised clustering algorithm including at least one of: K-means clustering, principle component analysis, or a latent space of an autoencoder; or a supervised method, such as a multilayer perceptron, a convolutional neural network, a transformer, or a recurrent neural network.

According to an embodiment, the output includes at least one of: a plurality of paths connecting elements of interests of the sample ribosome structure; nodes that appear the most often in the plurality of paths; residuals associated with the output characterizing the interaction of elements of the sample ribosome structure for local targeting; or a subset of the plurality of paths susceptible to therapeutic targeting.

According to an embodiment, the processing device is configured to present the output by: modeling an agent as one or more nodes of the network graph; and determining the agent as active when at least one of the following conditions is satisfied: a number of edges has been changed exceeding a threshold; a path connecting predefined regions of interest of the ribosome has changed; or a difference between the sample ribosome structure and a reference ribosome structure exceeds a threshold value.

### BRIEF DESCRIPTION OF THE DRAWINGS

The invention will be better understood upon reading of the following description, which is given solely by way of example and with reference to the appended drawings, in which:

FIG. 1: Schematic representation of the calculation of the isolated solvent surface accessible area. (a) Schematic representation of the calculation of the isolated solvent surface accessible area of two nodes (node γ and node B) separately. (b) Schematic representation of the calculation of the isolated solvent surface accessible area of the pair of nodes (pair comprising node γ and node B) in the absence of interaction. (c) Schematic representation of the calculation of the isolated solvent surface accessible area of the pair of nodes (pair comprising node γ and node B) of two nodes interacting. If the isolated solvent surface accessible area of the pair of nodes is smaller than the sum of the two isolated solvent surface accessible area of said nodes in isolation, an interaction is found. The grey spheres with black center represent areas occupied by the pseudo probe solvent molecules. The radius of the pseudo probe solvent is taken to be the radius of the solvent molecule (1.4 Å)+the mean displacement radius of both nodes. In (b) the sum of Aγ and A_{B} is the same as A_{Y+B} and there is no interaction. (c) An interaction is found because the sum of Aγ and A_{B} is larger than A_{Y+B}.

FIG. 2: Graphical representations of a ribosome. On the left side: classical structural representation of a bacterial ribosome; on the right side: interaction network representation according to the invention of the bacterial ribosome based on pdb 1vy4, from Polikanov et al (2014) Nat Struct Mol Biol 21 787. The square nodes indicate rRNAs. The circle nodes are the secondary structure element of either 16S or 23S. The light grey indicates the large subunit and the dark grey the small subunit. The triangles indicate rproteins. The position of the nodes represents the center of mass of the element from the pdb file.

FIG. 3: Interaction network representations according to the invention plotted as a two-dimensional graph of ribosomes of Escherichia coli in three states. (a) Classical state. (b) With paromomycin. (c) With mRNA stem-loop. Peptidyl transferase center (PTC) and decoding center (DC) are identified and paths with maximum of three nodes comprising them are in bold.

FIG. 4: Organigram of a method according to the invention, for identifying the region targeted by an active agent on a complex molecular structure or for identifying the activity of an agent on a complex molecular structure.

FIG. 5: Histogram of E. coli's ribosome connectivity from 197 structural files with a resolution inferior to 4 Å. The solid line is a fit to a Gaussian distribution having mean u and standard deviation o as indicated in the legend. The files falling within the Gaussian, with lower number of connections, correspond to ribosomes with greater degrees of freedom, which are necessary for the elongation process. Files to the right of the Gaussian or falling outside comprising the second peak (circled), correspond to ribosomes with fewer degrees of freedom due to the extra interactions. Elongation is typically more difficult in such ribosomes.

FIG. 6 illustrates a block diagram of a computer system operable to perform various example operations herein, according to aspects of the present invention disclosure.

FIG. 7 illustrates a flow chart of an example method of calculating a sample ribosome structure based on a machine-learning (ML) model, according to aspects of the present invention disclosure.

FIG. 8: illustrates a flow chart of an example method of calculating a sample large complex biomolecule based on a machine-learning (ML) model to validate whether a candidate drug compound is effective on the sample large complex biomolecule, according to aspects of the present invention disclosure. FIG. 6: Interaction networks representations provided to and reconstructed by the deep autoencoder employed in example B. The left images (pdb 7st6) correspond to the ribosome in the classic state (also shown in FIG. 3a). Its reconstructed image has few mistakes (depicted in black lines for new edges and dashed lines for edges in the initial graph not present in the reconstructed graph). There were 94 errors out of 3868 features. The middle figures shows the initial and reconstructed interaction network representations of the structural pdb file 7k00, which is a classic state but in the presence of paromomycin, corresponding to FIG. 3b. This interaction network representation was observed visually to be significantly different from the classic state without antibiotics (left figure). Using the autoencoder, we find the reconstruction has 677 errors out of 3868 features. More subtly, the ribosomal structure in the classic state in the presence of the antibiotic Avilamycin shows (pdb 5KCR, right images) shows 396 errors out of 3864 features in the reconstruction. These images show how anomaly detection using an autoencoder can systematically detect subtle differences that are statistically significant between ribosomes with and without therapeutics when they are nominally in the same ribosomal state.

### DETAILED DESCRIPTION

The method according to the invention, preferably a computer implemented method, aims at identifying at least one biological characteristic of a complex molecular structure, in particular from its structural file. For example, the method enables for the determination of the therapeutical target on the complex molecular structures, similarity of two configurations of a complex molecular structure, region targeted by an active agent on the complex molecular structure, activity of an agent on the complex molecular structure, and identifying the specificity of an agent toward complex molecular structures.

Complex molecular structures according to the invention are complex molecular associations comprising at least 15 molecules, preferably at least 20 molecules and most preferably at least 30 molecules. These structures preferably perform complex reactions. Complex molecular structures of the invention can be from various origin, synthetic, from eukaryote or prokaryote organisms, or from viruses, in particular it can be of bacterial or eukaryote origin.

According to an embodiment the complex molecular structure is a nucleoprotein. Nucleoproteins are complex structures comprising proteins and nucleic acid (such as DNA or RNA). Nucleoproteins include for examples ribosomes, nucleosomes and viral nucleocapsids. More preferably complex molecular structures according to the invention are ribosomes.

The ribosome decodes the mRNA to assemble the amino acids into a polypeptide chain, which then folds into a functional protein. This process is named translation. The fundamental components of translation are ribosomes, mRNAs, tRNAs, and amino acids. tRNAs deliver each amino acid to the ribosome where the amino acid is attached to the polypeptide chain. Translation is split into three stages: initiation, elongation, and termination. During initiation, the ribosome subunit forms a complex to begin elongation. During elongation which occurs as a cycle, the ribosome translocates the mRNA in the 5′ to 3′ direction; at this stage, the ribosome synthesizes a growing polypeptide chain using aminoacyl-tRNAs (aa-tRNA). Finally, termination is when the ribosome recognizes a release factor at the stop codon and release the polypeptide.

Complex molecular structures according to the invention preferably perform several enzymatic activities. Complex molecular structures can be found in various configurations, some of which corresponding to nonfunctional state and some of which corresponding to functional state. In a non-functional state, the complex molecular structure stop performing complex reactions. For example, ribosomes can be found in different configurations corresponding to: each step of the elongation cycle, or its assembly, or hibernation state, or interacting with an active agent (antagonist or agonist), etc.

By “biological characteristic”, it is meant a characteristic with relevance to the biological fields, such as: determining therapeutical target on the complex molecular structure, the configuration of the complex molecular structure, the similarity of two configurations of a complex molecular structure, the region targeted by an active agent on the complex molecular structure, activity of an agent on the complex molecular structure and identifying the specificity of an agent toward complex molecular structures.

By “agent” it is meant a molecule that is tested on the complex molecular structure. Typically the agent is chosen among antibiotics, anti-viral drug, anti-cancer drug, therapeutics, drug candidate, antimicrobials, small molecules, antibodies and fragment thereof.

By “the region targeted by an active agent” it is meant the region or region(s) in the complex molecular structure that interact with the agent.

By “the activity of an agent” it is meant the effect of the agent on the activity of the complex molecular structure. For example the agent can be an agonist or an antagonist activity of the complex molecular structure.

According to the invention, “predefined regions of interest” of the complex molecular structure are region of the structure already known to be implicated in important activity of said structure. For example, in the ribosome predefined regions of interest includes the peptidyl-transferase center (PTC) and the decoding center (DC).

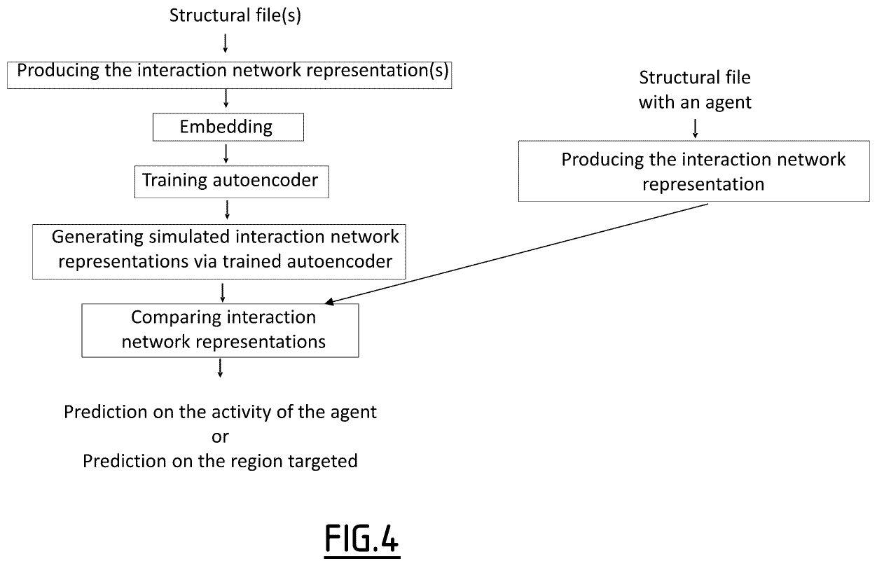

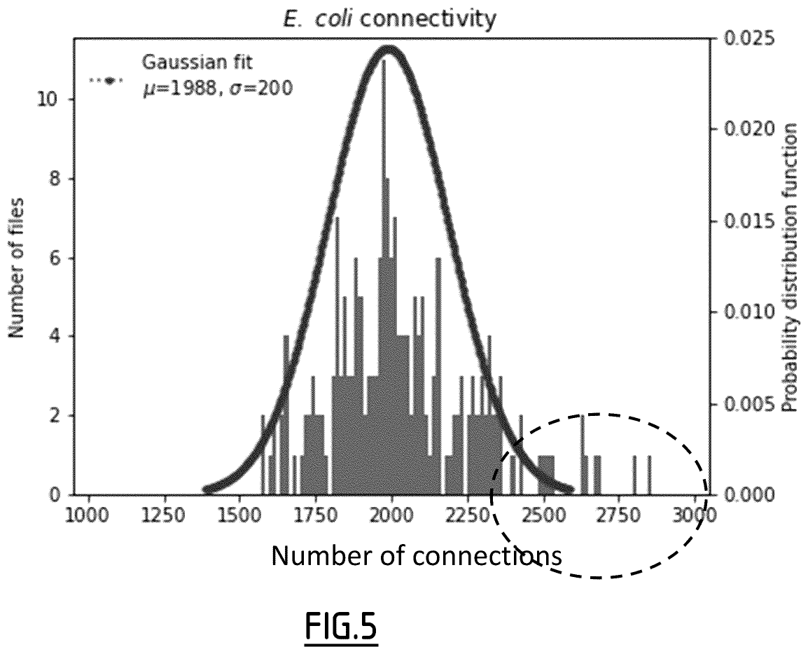

A structural file, shown as input in the organigram of FIG. 4, and in particular structural file in the context of the invention, is a file comprising 3D structure of a complex molecular structure, and in particular atomic coordinates of molecules. Structural files of complex molecular structures are available in several banks of data such as the ProteinData Bank. These files can be in Protein Data Bank (PDB) file format or mmCIF format. The PDB format provides description and annotation of protein and nucleic acid structures including atomic coordinates, secondary structure assignments, as well as atomic connectivity.

This structural file can be obtained from several methods, including X-ray crystallography, Nuclear magnetic resonance (NMR) spectroscopy, and cryo-electron microscopy (cryo-EM). The results of these methods allows the scientist to build a representation that is consistent with both the experimental data and the expected composition and geometry of the molecule. The results of several methods can also be combined to sort out the atomic coordination, this practice of fusing multiple experimental approaches is often referred to as Integrative or Hybrid Methods (I/HM).

Structural file also includes b-factors values. B-factor describes the displacement of the atomic positions from an average (mean) value (mean-square displacement). Higher flexibility results in larger displacements and, eventually, lower electron density. For x-ray diffraction or NMR spectroscopy, the b-factor corresponds to Debye-Waller factor and describes the attenuation of the X-ray or neuron scattering caused by thermal motion. For cryo-EM, b-factors are determined during image formation from the Gaussian envelope function, but can also be used to refine the models, often resulting in a global value, although there are techniques to determine local values.

Nodes according to the invention represent either a molecule or a part of a molecule of the complex molecular structure, or in some embodiments, the agent tested with the complex molecular structure. Preferably, according to the invention, each node is determined as a functional structure of the complex molecular structure. Indeed complex molecular structures have been studied for years and several functional structures have been found. For example, in ribosome, helices H74 and h44, where capital H indicates a secondary structure from the large subunit 23S rRNA and the lowercase h indicates a secondary structure from the small subunit 16S rRNA, are known for having special functions. H74 is a part of the peptidyl-transferase center (PTC) and h44 is a part of the decoding center (DC).

When the complex molecular structure is represented with an agent, the agent does not necessarily need to be represented as a node. If the agent is bound to a node then the agent is considered to be part of that node and the interactions of that node can be a result from either the agent or the other part of the node. In such cases, the agent is typically a very small molecule and has been found to typically just form connections with the node. In this case, the changes in the connections of the node in response to the agent and the secondary effect on other nodes in the network are the dominant source of changes of the network. Another case is when the agent is added to the enumeration of residues in a structural element of the structural file and the elements of the network for that structural element use the residues of the structure to determine the nodes of the network representation. This is the case for an agent added for instance to the enumeration of the residues of 16S or 23S rRNAS of a ribosome. In this case, the ‘residues’ corresponding to the agent can be isolated and made into a separate node in the interaction network representation. Finally, the agent can also be represented in the structural file as a separate structure and in this case the agent can be used directly as a node in the interaction network representation.

In another embodiment each node is determined according to secondary structures present in the complex molecular structures.

Secondary structures are particular local spatial conformations of a polypeptide or a nucleic acid. The most common secondary structures for proteins are alpha helices, beta sheets, beta turns and omega loops. The most common secondary structures for nucleic acid are double helices, stem loop and pseudo knot.

For representations done on different structural files of complex molecular structures comprising nucleic acid and/or proteins to be comparable, and the most accurate, the decomposition of the proteins and nucleic acid into secondary structure needs to be aligned to a reference. In other words, the decomposition of the secondary structure is preferably done using known annotation. This step ensures that the nodes are identically determined.

For example, the decomposition of the secondary structure of a ribosome is preferably done using the published annotation found in the ribovision website.

In a particular embodiment, the nodes are determined according to the following rules:
1. each secondary structure of each protein is defined as a respective node,
2. each peptide is defined as a respective node,
3. each nucleic acid of less of 1000 nucleotides is defined as a respective node,
4. each secondary structure of each nucleic acid of more of 1000 nucleotides is defined as a respective node.

Peptides, according to the invention, comprise from 2 to 100 amino acids. Proteins, according to the invention, comprise more than 100 amino acids.

In the case of long secondary structure containing different functional parts, such as for example h44, said secondary structure can be further decomposed into smaller sections corresponding to each functional part, defined each as a respective node.

In a particular embodiment, when the complex molecular structure is a ribosome, the nodes can be determined according to the following rules:
1. each ribosomal protein (r-protein) is defined as a respective node,
2. each rRNAs having a sediment coefficient of Svedberg superior to 10 (i.e. superior to 10S) is defined as a respective node,
3. other rRNAs are each defined as a respective node,
4. the mRNA is considered a respective node, and
5. each tRNA is considered a respective node.

Typically, the Shrake Rupley method is employed to calculate the isolated solvent surface accessible area (or solvent-accessible surface area (SASA)) of a node.

For proteins and nucleic acid, this method is done at the level of each residue in the node and the SASA for each residue are then summed. The SASA for each residue proceeds by calculating for each atom in the residue, the total SASA for the atom.

1) A sphere centered at each atom a is determined where its radius Ra is the sum of the van der Waals radius of that atom and the radius of a pseudo probe molecule R_{probe}
2) 100 points are distributed on this sphere, using for, instance the Golden Section spiral algorithm.
3) The SASA of an atom is the area on the surface of a sphere of radius R, on is then approximated by the sum of all atoms where the probe molecule placed in contact with this atom without penetrating any other atoms of the molecule:

$$SASA = \sum \frac{R}{\sqrt{{R}^{2} - {Z}_{i}^{2}}} D {L}_{i}$$

with D=ΔZ/(2+Δ′Z),
where Lᵢ is the length of the arc drawn on a section i and Zᵢ is the perpendicular distance from the center of the sphere to the section i, ΔZ is the spacing between the sections and Δ′Z is the smaller of ΔZ/2 and R−Zᵢ.

This is the standard method for calculating the SASA, taken directly from the paper by Shrake Rupley, where the calculation originates from Lee and Richards. Usually, the probe solvent taken for this calculation is water molecule with a radius of r_{H20}=1.4 Å. However, the inventors choose to use a pseudo probe solvent molecule which takes into account the b-factor of said node in the structural file.

Thus, according to an embodiment of the invention, in step ii) and iii), the b-factor of said node(s) in said structural file is taken into account to calculate the radius of the pseudo probe solvent molecule.

The b-factor of a node according to the invention is:
the average of the b-factors of the alpha-carbon atom of each amino acid of a node composed of a protein,
the average of the b-factors of the phosphate atom of each nucleotide of a node composed of nucleic acid, or
If the node is neither a protein nor a nucleic acid, the average b-factor of all the atoms in the molecule. In such a case, if the molecule is a small molecule, for instance a therapeutic, and it is likely that the atoms would have similar b-factors. In other cases, the average b-factor should exclude parts of the molecule prone to thermal vibrations which can induce anomalously large b-factors. The total value for the pseudo probe molecule should in general be smaller than 10 Å and never exceed 15 Å to avoid errors in the calculations of the interactions.

Preferably, the radius of said pseudo probe molecule for node with index i, is calculated as follows:

$${R}_{probe} ( {node}_{i} ) = {r}_{H 2 O} + {d}_{nodei}$$

wherein R_{probe}(nodeᵢ) is the radius of the pseudo probe for the nodeᵢ,
r_{H20} is the radius of water molecule, and
d_{noder} is the mean displacement of the node with index i.

Preferably, the radius of a pseudo probe molecule for the pair of nodes with indexes i and j, is calculated as follows:

$${R}_{probe} ( {pair}_{ij} ) = {r}_{H 2 O} + {d}_{nodei} + {d}_{nodej}$$

wherein R_{probe}(pairᵢⱼ) is the radius of the pseudo probe for the pair of nodes with indexes i and j,
r_{H20} is the radius of water molecule,
d_{nodei} is the mean displacement of the first node with index i of the pair, and
and d_{nodej} is the mean displacement of the second node with index j of the pair;
wherein the mean displacement is given by the relation:

$${d}_{n o d e i} = \sqrt{\frac{3 {B}_{i}}{8 {π}^{2}}}$$

wherein Bᵢ is the b-factor of the node with index i.

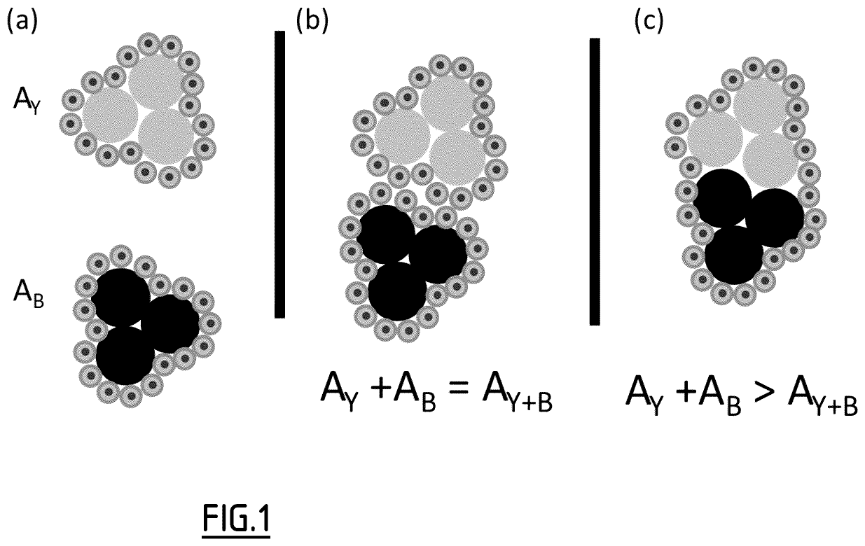

Once the SASA of each node is calculated individually, their sum is found (SASAᵢ+SASAⱼ). Next the elements are isolated together as a pair and the SASA of the pairᵢⱼ is calculated (SASAᵢ₊ⱼ). In other words, instead of calculating the SASA for both nodes, it is calculated for the two of them at the same time. So, if atoms of the nodes are in contact with each other, the calculated SASAᵢ₊ⱼ will be smaller than the sum SASAᵢ+SASAⱼ (see FIG. 1).

According to the invention, an interaction network representation, obtained during the step “Producing the interaction network representation” shown in FIG. 4 and as described above, comprises nodes connected by edges. Thus, a path can be defined from this representation. Paths according to the invention are constituted of connected nodes and edges, each path comprising at least two nodes, more preferably each path comprising less than 10 nodes, more preferably less than 5 nodes.

A node or an edge according to the invention can also have an attribute, for example simplifying its identification or analysis. Such attribute can be for example the name of the functional part of the node, the name of the particular region represented by the node, the configuration of the ribosome, the presence of a mutation in a nucleic acid or in a protein, the type of the node (amino acid sequence, nucleic acid or else), a probability of presence of the edge, etc.

Preferably the interaction network representation obtained by the method of the invention allows to obtain at least one parameter, such as the number of edges, an average degree or connection of the nodes, an average path length of the network graph, a density of the network graph, etc.

The inventors have shown that agents that arrest the elongation process of ribosome tend to have interaction representation with very high connectivity. Very high connectivity is also observed when the ribosome is in a hibernating or frozen configuration. Thus, the interaction network representation of the invention can be used to identify at least one biological characteristic of the complex molecular structure.

Advantageously, the information from the interaction network representation, also called graph, is transformed into a matrix representation, this transformation being also called “Embedding”, as shown in FIG. 4. Such matrix representation is for example in the form of a matrix of rows of length N, where N is the total number of elements in all of the set of graphs and each row represents a single element in the set of all possible elements. The columns could represent local graph properties, these can be in terms of a larger dimensional space, such as a vector space containing each of this information, or just a single value.

Hereinafter are some examples of embedding:

### A. Direct Connectivity

Each column represents every node in the graph, i.e. in the interaction network representation, and the value entered corresponds to the minimum distance between the node in the row and the node in the column, so that ‘0’ represents that the node in the row and the node in the column are the same, and ‘1’ represents an edge between the node in the row and the node in the column, larger values represent the smallest path length between the node in the row and the node in the column and if the two nodes are not connected then the value would be indicated by N+1. To reduce the dimensionality of the direct connectivity, connections present in the train set can be condensed into a vector and used as input into the algorithm. Such an embedding is used in Example B.

B. Residue Connectivity (Information about the Interactions)

Each column is assigned an additional vector symbolizing all of the residues involved for each node. This indicates, for example, the residues for the node in the column that are involved in all the interactions that were observed in the graph, i.e. the interaction network representation. For instance, for protein ul4, one would list all of the resides that are involved in all the interactions where uL4 is involved and then for these interactions a ‘1’ or a ‘0’ would be added to correspond whether or not the residue is involved. This could be used for instance to construct a bipartite graph was envisioned where the graph consisted of a set of nodes for the elements and set of nodes for the interactions.

### C. Local Structure

Each column vector represents for example one of a series of characteristics of the node relating to the structure of the graph, i.e. of the interaction network representation, that is important. These characteristics could be:
i. The interaction network representation could be divided into communities using methods such as by modularity measures, k-means, Girven-Newman algorithm, etc. Communities are groupings of the nodes as determined by their neighborhood of connections. In this case, the vector description could be very simplified with each node identified by its community. One could break the graph up (via an algorithm) into 5 communities and then identify each community by a particular functional center (for instance tRNAs, the decoding center, the peptidyl transferase center). The characteristic for this particular column would be a number corresponding to a community.
ii. The columns could represent the distance (smallest path length) to important functional centers (for instance the PTC, DC, tRNAs, etc.)
iii. The columns could represent the number of path lengths to important functional centers (for instance 5 paths between the PTC and the DC of length 2) iv. The columns could represent nodes where the nodes are involved in the paths between functional centers

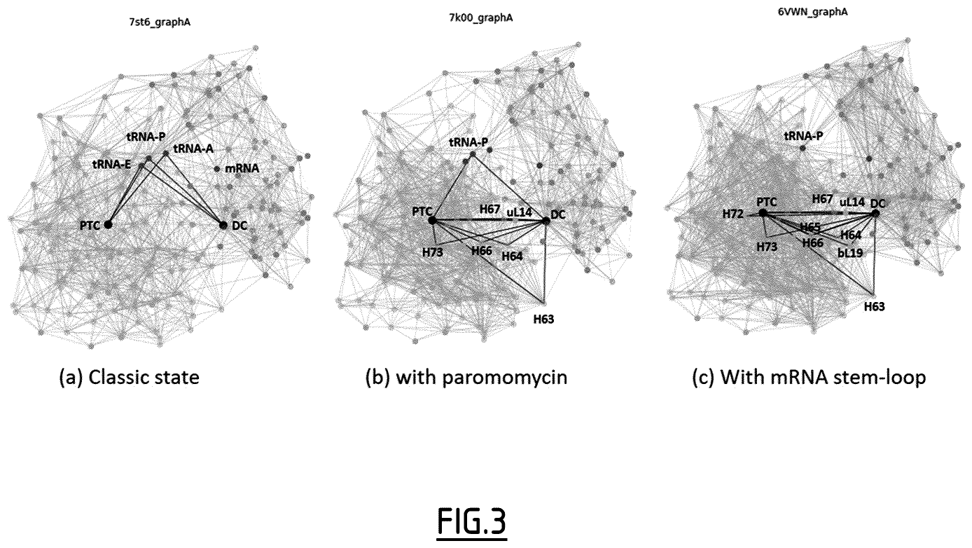

Advantageously, the embedding thus allows the representation of the most important structural features of each interaction network representation depending on the desired result. For instance, in the case of a ribosome, it may be desirable to determine which residues are involved in the additional connectivity exhibited in presence of an antibiotic, as for instance in FIG. 3b. This would allow understanding of how a particular therapeutic is interacting with the ribosome and how it affects connectivity. Specifically, the determination of which residues in nodes other than the ones connected to the therapeutic are the most susceptible to additional connectivity impeding ribosome function could potentially be used as a target for not yet considered therapeutics.

According to an embodiment of the invention, the method comprises:
a) producing several interaction network representations of a complex molecular structure in different configurations according to step S1),
b) training an autoencoder, wherein said autoencoder comprises an encoder, a decoder connected at output of the encoder, wherein the output of the encoder is a latent space, and wherein the output of the decoder is similar to the input of the encoder, wherein interaction network representations of step a) are used as a training dataset of said autoencoder, and preferably
c) producing, as output of the trained autoencoder, simulated interaction network representations of said complex molecular structure, different simulated interaction network representations being produced by adapting respective parameters in the latent space.

In an encoding phase, the encoder is configured to reduce the dimensionality of the input into a latent vector. Said latent vector is reduced in dimensionality compared to the input. The encoder according to the invention can be chosen among: a convolutional neural network, and long short term memory neural network (e.g. a recurrent neural network) or a multi-layer perceptron.

Then, in a decoding phase, the decoder is configured to perform the inverse operations of the encoder. The resultant output of the decoder is an estimation of the input of the encoder. The difference between the input and output is known as the reconstruction error.

In the training phase, corresponding to the step “Training autoencoder” shown in the organigram of FIG. 4, the reconstruction error is minimized by defining a loss function and adjusting the weights in the layers of the autoencoder, in particular the weights in the layers of the decoder, so that the loss function is minimized. The skilled person will observe that minimizing the reconstruction error allows having the output of the decoder is similar to the input of the encoder. In other words, the output of the decoder is similar to the input of the encoder by minimizing the difference between the input of the encoder and the output of the decoder, typically by minimizing the loss function depending on said difference. Typical loss functions are cross entropy, mean square error, etc. At the end of the training, the final reconstruction error is low. After sufficient training, the output of the decoder should resemble the input of the encoder for all of the graphs used for the training. The difference between input and output of the final autoencoder is known as the ‘reconstruction error’.

Simulated interaction network representations, also named estimated interaction network representations, can be produced by this method of the invention. Indeed, this method allows the extrapolation of configurations for which not structural files are available, for example because of technical obstacles.

In a preferred embodiment, interaction network representations are “embedded” during the aforementioned step “Embedding” shown in FIG. 4, before being used as an input of an autoencoder.

The latent space can also include the constraint that it follow a probability distribution, preferably a normal distribution with as parameters mean and standard deviation.

According to an embodiment of the invention, the autoencoder is trained with a training dataset corresponding to a particular series of configurations of the complex molecular structure, during the step “Training autoencoder” shown in FIG. 4. For example, the method can comprise:
training an autoencoder with interaction network representations of ribosomes in the process of the elongation, and/or
training an autoencoder with interaction network representations of ribosomes in the process of the elongation where the representation of the ribosome is restricted only to the ribosome in its classic state, and/or
training an autoencoder with interaction network representations of ribosomes in the process of the elongation where the representation of the ribosome is restricted only to the ribosome in its hybrid state, and/or
training an autoencoder with interaction network representations of ribosomes in the process of the assembly, and/or
training an autoencoder with interaction network representations of ribosomes in the hibernation state, and/or
training an autoencoder with interaction network representations of ribosomes during termination or recycling, and/or
training an autoencoder with interaction network representations of ribosomes with an antibiotic present on the ribosomal structure. Then, a new interaction network representation of the complex molecular structure is used as input of one autoencoder, and from the reconstruction error, it can be determined if said new interaction network representation is part of the series used as the training data set, or not.

According to an advantageous aspect of the invention, for example during a step “Comparing interaction network representations” shown in FIG. 4, the similarity between two interaction network representations is evaluated from a distance between these two interaction network representations, and the lower this distance is, the higher the similarity is. In other words, the similarity between two interaction network representations corresponds to the inverse of this distance between these two interaction network representations. It can be evaluated for example from the distance between these two interaction network representations from their output in the latent space and/or from the distance from their output of the decoder.

This distance is preferably of the type chosen among the group comprising:
distance according to the Minkowski norm,
mean square error and
cosine distance.

According to an embodiment of the invention, the identified biological characteristic is the similarity of two configurations of a complex molecular structure, wherein said method comprises:
a) producing several interaction network representations of said complex molecular structure in different configurations according to step S1,
b) training an autoencoder, wherein said autoencoder comprises an encoder, a decoder connected at output of the encoder, wherein the output of the encoder is a latent space, and wherein the output of the decoder is similar to the input of the encoder, wherein interaction network representations of step a) are used as a training dataset of said autoencoder,
c) using as input to the autoencoder interaction network representations of the two configurations to compare,
d) determining a similarity between the two configurations from their output in the latent space and/or from their output of the decoder.

In a particular embodiment of this method, the identified biological characteristic is the similarity of two series of configurations of a complex molecular structure. Then in step c), interaction network representations of the two series of configurations are used as input in the autoencoder, and in step d) the similarity between the two configurations series from their output in the latent space and/or from their output of the decoder is determined.

According to an embodiment of the invention, the identified biological characteristic is the specificity of an agent toward complex molecular structures comprising:
a) realizing the method to identify the activity of the agent as defined above on a first complex molecular structure,
b) realizing the method to identify the activity of the same agent as defined above on a second complex molecular structure, and
c) when the agent is active on only one complex molecular structure, concluding that said agent is specific to said complex molecular structure.

According to a particular embodiment of this method, the first and the second complex molecular structures are homologous, but of different origin. Then this method allows to identify specific agents.

As used herein the term homologous regarding two complex molecular structures means two complex molecular structures having shared ancestry in the evolutionary history of life. Often these homologous complex molecular structures perform the same or similar complex reaction(s) in different organisms.

According to a particular embodiment of this method, the first and the second complex molecular structures perform the same complex reactions.

This method can be very valuable for finding new antibiotics or antimicrobial. For example, if an agent is active on a prokaryote ribosome and put it in a non-functional state but does not have activity on a eukaryote ribosome, then it is a promising agent as an antibiotic.

The subject-matter of the invention is also a computer program comprising software instructions which, when executed by a computer, implement a method as defined above.

### EXAMPLES

### Example A: Study on the Ribosomes of E. coli

To illustrate the method, the ribosomes of E. coli were utilized. The first step was to identify useful structural files. An initial search of the protein databank (rcsb.org) found all E. coli ribosomal structural files with a resolution inferior or equal to 4 Å. Structural files whose resolution was greater than 4 Å were also included if the number of interactions were comparable and the file originated from a publication containing files with a resolution≤4 Å. The calculation was performed as follows. First, the ribosome was divided into constituent nodes that will be used to identify interactions. Each ribosomal protein (r-protein) and the mRNA are considered as single node. The smaller RNAs including tRNAs and 5S rRNAs were represented as a single node. The larger 16S and 23S rRNAs are divided according to secondary structures following the notation of the Ribovision (Ribosome Visualization Suite) website.

The computation of the interactions is done systematically whether or not an interaction is expected. For example, the pdb file 7st6 contains 213 nodes including 50 r-proteins, 158 secondary structures from 16S and 23S rRNAs and an mRNA, a 5SrRNA and 3 tRNAs. To determine the interactions, inventors loop over all possible ways to obtain an interaction involving

$$2 nodes ( \begin{matrix}213 \\ 2\end{matrix} ) ,$$

which results in 22,578 calculations, of which just 1939 interactions are found.

Using over 200 high resolution structural files of the Escherichia coli ribosomes, inventors found that the E. coli ribosomes representations have a mean of 1988+200 interactions (see FIG. 5) and that 90% of the representations contain the same 1251 interactions. The latter are termed ‘common interactions’, and expected to play a structural role, ensuring the ribosome stability during the dynamic motion that allows it to carry out its function. Each of these interactions can be assigned a probability that can serve as a comparison for its presence or absence in other species. In FIG. 5, files with fewer connections correspond to ribosome with greater degrees of freedom and more likely to be able to proceed through the elongation process. Files with larger number of connections (circled), have fewer degrees of freedom. The presence of additional interactions can restrict its freedom of motion and inhibits it from proceeding along the path of elongation. Some of the states with larger number of connections can correspond to hybrid states. However, many states correspond to hibernating states or the presence of an element outside of normal elongation steps. For instance, in FIG. 3c, an mRNA with a stem-loop which shifts the reading frame of the ribosome and is a common feature in viruses, results in 25% greater connectivity compared to the classic state in FIG. 3a. This extra connectivity significantly reduces the ribosomes range of motion and restricts its possibilities. It also serves as a method to detect anomalous behaviors, either due for instance to a virus hijacking the ribosome to produce its own proteins or due to the presence of a therapeutic that is responsible for the arrest of elongation. These results show how the methods of the invention can allow a fast screening of candidate drug without biological testing.

Looking more closely at the different graphs, the inventors observed that the global graph properties are very similar (e.g. average path length, diameter, average number of connections per node), but local connectivity can vary depending on the ribosomal state and the presence of a chemical or biological agent (see FIG. 3). Paromomycin is an antimicrobial which inhibits protein synthesis known to bind to h44, at the decoding center (DC). The gag-pol transcript of Human Immunodeficiency Virus (HIV) contains a stem loop causing frameshifting of the ribosome. In FIG. 3, the representations are plotted by determining the center of mass coordinates of each node and then projected into two-dimensions. In this figure, important changes can be seen in the paths between two important functional centers (PTC and DC). In the classic state only paths comprising 3 nodes or less are observed and only through the tRNAs. In the state with paromomycin, a direct path is observed as well as 6 additional path. Finally, when a frame-reading error mRNA is present on the ribosome, 8 additional path lengths are observed. The ribosome with the mRNA stem loop exhibits overall a much larger connectivity (2501 connections) than the other two ribosomes. Here, the increased connectivity represents a reduction in the possible motions the ribosome can realize. In the classic state, inventors found a value very similar to the mean (1939 connections). For the ribosome with paromomycin, they found fewer connections overall (1809 connections) but a denser connectivity at helices H73 and h44. The changes in structures b) and c) explain how translation is stopped or slowed from a small modification to the overall ribosomal structure. Connectivity reveals new insights not evident from a conventional structural representation.

### Example B: Use of a Deep Autoencoder for Anomaly Detection of Ribosomes in the Presence of an Antibiotic

The ‘educated’ observations between the different states in FIG. 3 can be made systematically by employing machine learning algorithms. To demonstrate this, inventors used an unsupervised anomaly detection with a deep autoencoder. In this bottleneck architecture, the latent space is a condensed representation of the initial input. If the autoencoder is trained with data from a particular ribosomal representation, the reconstruction of the input should represent a high fidelity estimation of the initial input. However, if after training, a ribosomal representation is introduced to the autoencoder that is sufficiently different from the trained data, then the reconstruction of its input will have a significant number of errors. This type of anomaly detection is considered ‘unsupervised’ because the anomalies are not learned by supervision. It can be made supervised by introducing labels into the initial training data. The unsupervised method is particularly relevant when the training data is limited or the labels of the anomalies are not known.

To demonstrate an anomaly detection using said autoencoder, inventors employed a standard deep autoencoder algorithm that is trained to recognize ribosomes in the classic state without the presence of antibiotics or other anomalies. From a database of 223 E. coli ribosomes with resolution inferior to 4 Å, for which the interaction network representations were calculated, 47 ribosomes were found to be in the classic state. Of these 47 ribosomes, 32 ribosomes were found to be in the process of elongation without antibiotics or anomalies. The auto-encoder was trained using these 32 ribosomes. The pdb references for these ribosomal structures are the following: 7n1p, 7n31, 7st2, 7st6, 6wd0, 6wd1, 6wd2, 6wd3, 6wd4, 6wd5, 6wd6, 6wd7, 6wd8, 6wd9, 6wda, 6wdd, 6wde, 6wdf, 6wdg, 3j9y,5mdz, 609j, 7nww, 7019, 7oif, 7oig, 7ot5, 8bgh, 8bil, 5uyk, 5uyl and 5uym.

Of the remaining 15 ribosomes, 9 (reference pdbs: 6wdh, 6wdi, 6wdj, 6wdk, 6wdl, 6wdm, 5uyn, 5uyp, 5uyq) contain a small non-cognate error between the mRNA and the tRNA present on the ribosome. Such errors are found to result in very small changes on the ribosome. The 6 remaining ribosomes contained antibiotics including: apramycin (pdb 7pjs), paromoycin (pdb 7k00), Myxovalargin B (pdb 8b7y), dirithromycin (pdb 6XZA), evernimycin (pdb 5kcr), avilamycin (pdb 5kcs).

The input of the algorithm consists of a vector of length 3868 corresponding to all elements (nodes and edges) of the interaction network representations and interactions present in the training set of 32 ribosomes. The encoder of the autoencoder consists of completely connected (dense) layers: an input vector of 3868, 2 hidden layers of size respectively 1024 and 512 and an output of size 64. The latent space is thus a vector with length of 64. The decoder performs the inverse operation of the encoder, consisting of two densely connected hidden layers of respective lengths 512, and 1024 respectively and an output layer of 3868 that permits the reconstruction of the input ribosome. The 32 ribosomal structures were trained on the autoencoder one at a time (batch size of 1) and 100 epochs were employed, meaning that the 32 structures were used to train the autoencoder 100 times. The loss function used was the mean square error. The results on the train data revealed on average 63 errors in the reconstructed ribosome out of the 3868 inputs or an average reconstruction accuracy of 98.4%. The 9 non-cognate mRNA/tRNA ribosomes had an average accuracy of 97%. The 6 ribosomes in the presence of antibiotics had an average accuracy of 89%. Examples of the initial and reconstructed ribosomes are shown in FIG. 9.

This result shows how what was observed by educated assumptions in FIG. 3 can be explored systematically using a machine learning algorithm. If one were to use a large database where different ribosomal and species states were labeled, a more complex machine learning algorithm would detect more subtle changes and enable high fidelity predictions.

The inventors have shown how ribosomal structures can be systematically represented using a coarse-grained network and demonstrated that this representation provides new insights into ribosome function. Most notably a tightly bound ribosome or region in the ribosome is a potential signature of loss of function/dysfunction. This methodology could be used to explore ribosome structures in an elegant efficient manner, to decipher its function, and evolution. This has also implications for the exploration of how potential therapeutics interact with the ribosome and their effectiveness.

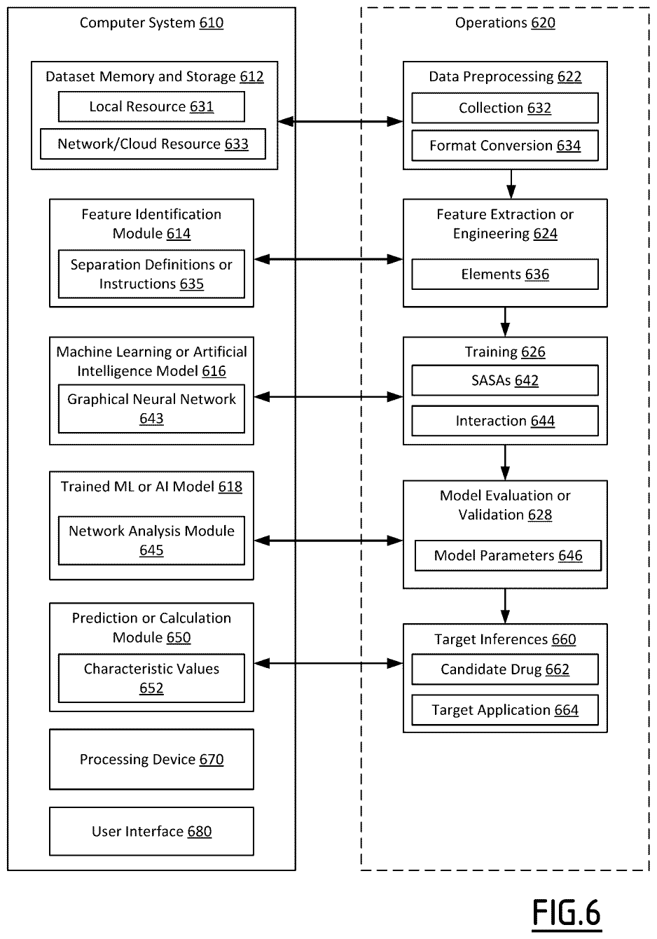

FIG. 6 illustrates a block diagram of a computer system 610 operable to perform various example operations 620 herein (including methods in FIGS. 1-6 above), according to aspects of the present invention disclosure. As shown, the computer system 610 includes a dataset memory and storage 612, a feature identification module 614, a machine learning (ML) or artificial intelligence model 616, a trained ML or Al model 618, a prediction or calculation module 650, a processing device 670, and a user interface 680. In aspects, the computer system 610 is configured to calculate a sample large complex biomolecule (e.g., a ribosome structure, as represented by one or more extracted elements 636) based on the trained ML or Al model 618 to validate whether a candidate drug 662 is effective on the sample large complex biomolecule. For example, a target set of compounds may first be identified based on one or more of: a defined target clinical application, a set of desired characteristics, or a defined class of compounds, which may be stored either in the local resource 631 or the network or cloud resource 633 of the dataset memory and storage 612. This identification process may be a portion of the data processing 622, in which data are collected at the collection 632 and/or converted at the format conversion 634.

The computer system 610 then pre-processes, such as by the feature identification module 614 and the ML or Al model 616, each compound of the target set of compounds to generate respective sets of feature data. The set of feature data may include a set of parameters based on a network analysis (e.g., as described in relation to FIGS. 3-5 above) for the ML model to predict interaction 644 of elements (e.g., based on SASAs 642). Model evaluation or validation 628 may be performed to associate one or more sets of model parameters 646 to one or more trained ML or Al models 618. The computer system 610 may process the sets of feature data with one or more trained ML models to produce predicted characteristic values 652 for each compound of the target set of compounds for each of the set of desired characteristics. The one or more trained ML models may be selected from a database of trained ML models based on at least the set of desired characteristics. The computer system 610 then identifies a subset of the target set of compounds based on the predicted characteristic values, as being effective on the sample large complex biomolecules.

The dataset memory and storage 612 may include non-transitory data storage devices and manage and facilitate interactions between various types of memory and storage devices, including, for example, Random Access Memory (RAM), Read-Only Memory (ROM), Hard Disk Drives (HDD), Solid State Drives (SSD), Hybrid Drives, Non-Volatile Memory express (NVMe), and Storage Class Memory (SCM). RAM may include dynamic RAM (DRAM), commonly used in computers for storing data in a separate capacitor within an integrated circuit; static RAM (SRAM), which is faster and uses multiple transistors per memory cell; and video RAM (VRAM), for graphics applications allowing simultaneous read and write operations. ROM may include programmable ROM (PROM), which can be programmed once post-manufacture; erasable PROM (EPROM), which can be erased with ultraviolet light; and electrically EPROM (EEPROM), erasable and reprogrammable through electrical charge. The local resource 631 may integrate non-transitory storage solutions like HDDs-magnetic storage devices known for high capacities- and SSDs, such as SATA, PCIe, and NVMe types. The network or cloud resource 633 may include network-attached storage (NAS) and cloud storage solutions, enabling efficient data access and synchronization across multiple platforms and geographical locations.

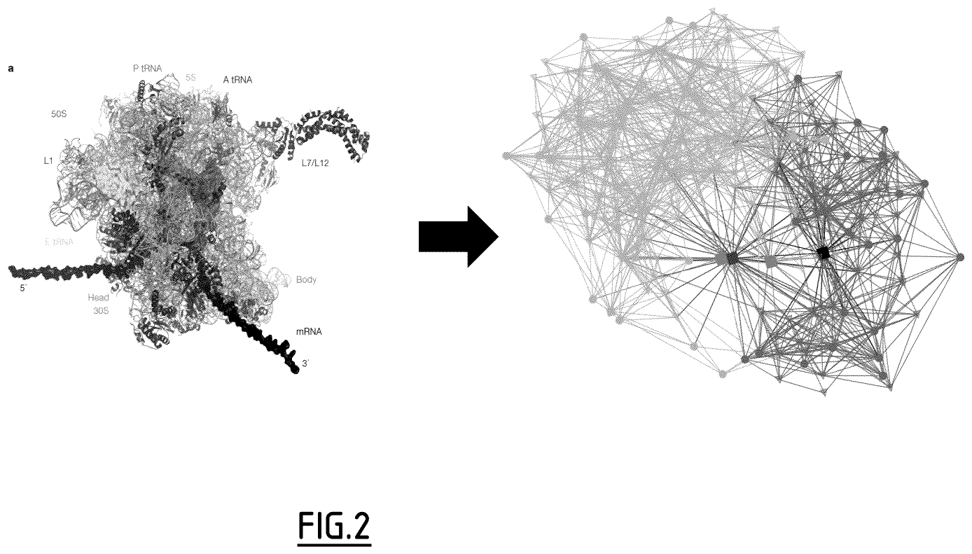

The dataset memory and storage 612 may include training dataset, validation dataset, test dataset, fine-tune dataset, operation dataset, and non-transitory storage medium for user input, including the sample large complex biomolecule. In some cases, the sample large complex biomolecule includes at least one of a ribosome or ribozyme including RNA and proteins, which are, via feature extraction or engineering 624 decomposable into at least one of secondary structures, tertiary structures, or domains defined in a use case (e.g., as shown in FIG. 2).

The feature identification module 614 includes feature definitions or instructions 635. For example, the separation definitions or instructions 635 enable the feature identification module 614 to separate data or dataset of ribosomal structures into constituent elements 636, including at least one of 5S rRNAs, tRNAs, mRNAs, ribosomal proteins, 16S rRNAs, or 23S rRNAs. The operations of the feature extraction or engineering 624 may further include representing the constitute elements 636 by a geometry that includes one or more perimeters (e.g., as shown in the examples related to FIG. 1).

The ML or Al model 616 includes a graphical neural network 643 and is configured to be trained by training operations 626. The training 626 may include receiving a training dataset of ribosomal structures and identify one or more single elements for each of the training dataset of ribosomal structures. The ML or Al model 616 calculates, in the network analysis (e.g., by the network analysis module 645), interactions of the one or more single elements using a difference between a sum of SASAs of the one or more single elements independently and an actual SASA of the one or more single elements together (e.g., as shown in FIG. 1). In some cases, the network analysis module 645 may compare, in the network analysis, the training dataset of ribosomal structures using either an unsupervised clustering algorithm including at least one of: K-means clustering, principle component analysis, or a latent space of an autoencoder; or a supervised method, such as a multilayer perceptron, a convolutional neural network, a transformer, or a recurrent neural network.

The ML or Al model 616 records a set of parameters 646 (e.g., using the trained ML or Al model 618) based on the network analysis for predicting the interaction of the elements of the sample large complex biomolecule. The model parameters 646 may be evaluated or validated by a model evaluation or validation operation 628 to result in one or more trained ML or Al models 618 (e.g., sorted or categorized based on various given conditions or origins). In some cases, the SASAs and SASA are computed using the Shrake Rupley method. Preferably, the radius of the solvent in said method should incorporate the b-factor of each node, using the pseudo solvent method described earlier.

For example, the model parameters 646 may include at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph; a cluster coefficient of the network graph; a density of the network graph; an assortativity of the network graph; a neighborhood of one of the number of nodes. The one of the number of nodes has a maximum degree exceeding a threshold or a reference value; paths between two or more of the number of nodes having a respective maximum degree exceeding the threshold or the reference value; a number of residues associated with an output of the ML model, the output characterizing the interaction of elements of the sample large complex biomolecule.

The prediction or calculation module 650 includes one or more characteristic values 652 produced, by performing target inferences 660 using the trained ML or Al model 618, for the candidate drug 662 and/or one or more target applications 664. For example, using the network analysis by the network analysis module 645 of the trained mL or Al model 618, the prediction or calculation module 650 may determine: for example, whether an antibiotic will be successful in reducing or arresting ribosome functionality, or whether a candidate drug 662 may or may not target at least a portion of a ribosomal structure based on the interactions (e.g., whether an intended action of a specific therapeutic targeting the ribosome is effective a particular therapeutic is effective in its action on the ribosome).

The processing device 670 is provided for interpreting and executing program instructions of the components mentioned above. The processing device 670 may manage data flow and perform the aforementioned computations. For example, the processing device 670 may include a Central Processing Unit (CPU), which handles arithmetical, logical, and input/output operations. CPUs may be single-core or multi-core. The processing device 670 may also include Graphics Processing Units (GPUs), specialized circuits to accelerate large volume computations, such as for images for display devices. The processing device 670 may also include Field-Programmable Gate Arrays (FPGAs) customizable post-manufacturing to fit specific applications, and Digital Signal Processors (DSPs) are tailored for real-time digital signal manipulation. The processing device 670 may also include Application-Specific Integrated Circuits (ASICs) performing dedicated functions in devices, enhancing efficiency in tasks such as data encryption and network processing. The processing device 670 may also include System on a Chip (SoC) architectures integrating components like CPUs, GPUs, and memory into a single chip, commonly used in mobile devices for their compact efficiency. The processing device 670 may also include Microcontrollers (MCUs) controlling specific functions in embedded systems such as automotive electronics and medical devices. The processing device 670 may also include cloud computing utilizing remote server networks to manage and process data, offering scalable resources and powerful computational capabilities without the need for local hardware investments.

The user interface 680 may include means through which a user interacts with the computer system 610. The user interface 680 may include graphical user interfaces (GUIs) featuring visual elements on devices such as personal computers, smartphones, and touch-enabled devices, to web-based interfaces accessed through browsers that include dynamic elements for interacting with web applications. The user interface 680 may include Command-line interfaces (CLIs) providing text-based interaction where users input commands directly. The user interface 680 may include Menu-driven interfaces guiding users through a series of menu choices. The user interface 680 may include voice user interfaces (VUIs) such as those in virtual assistants for interaction via spoken commands. The user interface 680 may include gesture-based interfaces detect physical gestures, used in VR systems and interactive installations. The user interface 680 may include natural user interfaces (NUIs) integrating gestures, touch, and voice for an intuitive user experience, such as in augmented reality applications that merge digital and real-world elements. In some cases, the output for validation or local targeting based on the interaction of the elements of the sample ribosome structure is presented via the user interface 680 of the computer system 610.

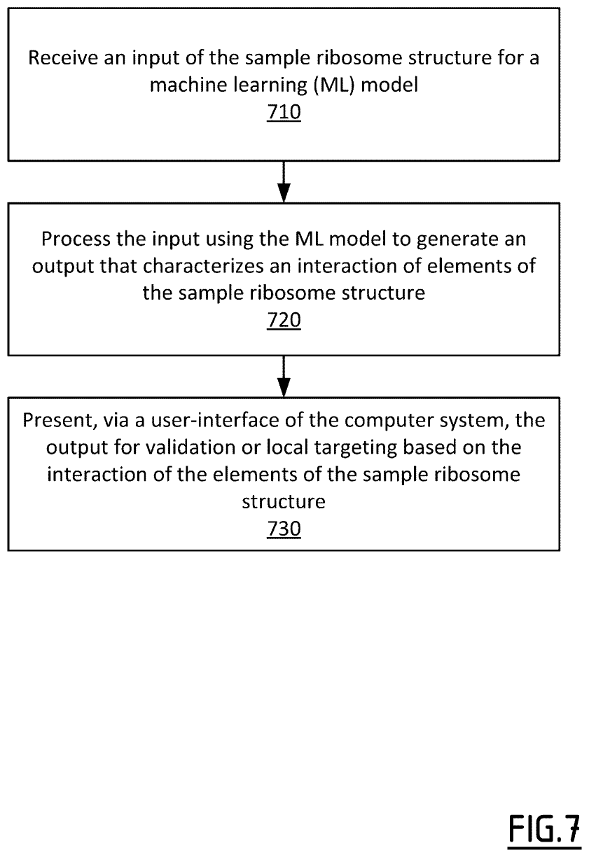

FIG. 7 illustrates a flow chart of an example method of calculating a sample ribosome structure based on a machine-learning (ML) model, according to aspects of the present invention disclosure. The example method starts by receiving 710, an input of the sample ribosome structure for a ML model. In some cases, the ML model has been trained using a training dataset of ribosomal structures by receiving the training dataset of ribosomal structures (e.g., FIGS. 2 and 5). One or more single elements are identified for each of the training dataset of ribosomal structures (e.g., as shown in FIG. 2). The interactions of the one or more single elements are calculated in a network analysis (e.g., as shown in FIGS. 3-5) using a difference between a sum of SASAs of the one or more single elements independently and an actual SASA of the one or more single elements together (e.g., as shown in FIG. 1). The ML model then records a set of parameters based on the network analysis for the ML model to predict the interaction of the elements of the sample ribosome structure.

The example method further includes processing 720 the input using the ML model to generate an output that characterizes an interaction of elements of the sample ribosome structure. The output is presented 730 via a user-interface of the computer system (such as the user interface 680 of the computer system 610) for validation or local targeting (e.g., the operations of target inferences 660) based on the interaction of the elements of the sample ribosome structure.

In aspects, identifying the one or more single elements for each of the training dataset of ribosomal structures includes: separating each of the training dataset of ribosomal structures into constituent elements, including at least one of: 5S rRNAs, tRNAs, mRNAs, ribosomal proteins, 16S rRNAs, or 23S rRNAs; and representing the constituent elements by a geometry having one or more perimeters. In some cases, the SASAs and SASA are computed using Shrake Rupley method. Preferably, the radius of the solvent in said method should incorporate the b-factor of each node, using the pseudo solvent method described earlier.

In aspects, the method further includes comparing, in the network analysis, the training dataset of ribosomal structures using either an unsupervised clustering algorithm including at least one of: K-means clustering, principle component analysis, or a latent space of an autoencoder; or a supervised method, such as a multilayer perceptron, a convolutional neural network, a transformer, or a recurrent neural network.

In aspects, the ML model includes a graphical neural network and wherein the set of parameters based on the network analysis for the ML model includes at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph; a cluster coefficient of the network graph; a density of the network graph; or an assortativity of the network graph.

In aspects, the output includes at least one of: a plurality of paths connecting elements of interests of the sample ribosome structure; nodes that appear the most often in the plurality of paths; residuals associated with the output characterizing the interaction of elements of the sample ribosome structure for local targeting; or a subset of the plurality of paths susceptible to therapeutic targeting.

In aspects, the presenting the output includes modeling an agent as one or more nodes of the network graph; and determining the agent as active when at least one of the following conditions is satisfied: a number of edges has been changed exceeding a threshold; a path connecting predefined regions of interest of the ribosome has changed; or a difference between the sample ribosome structure and a reference ribosome structure exceeds a threshold value.

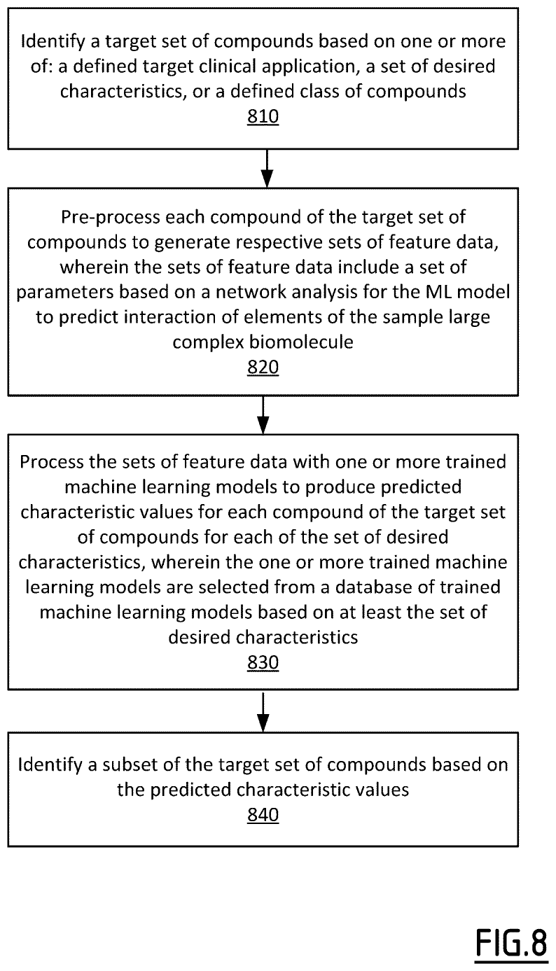

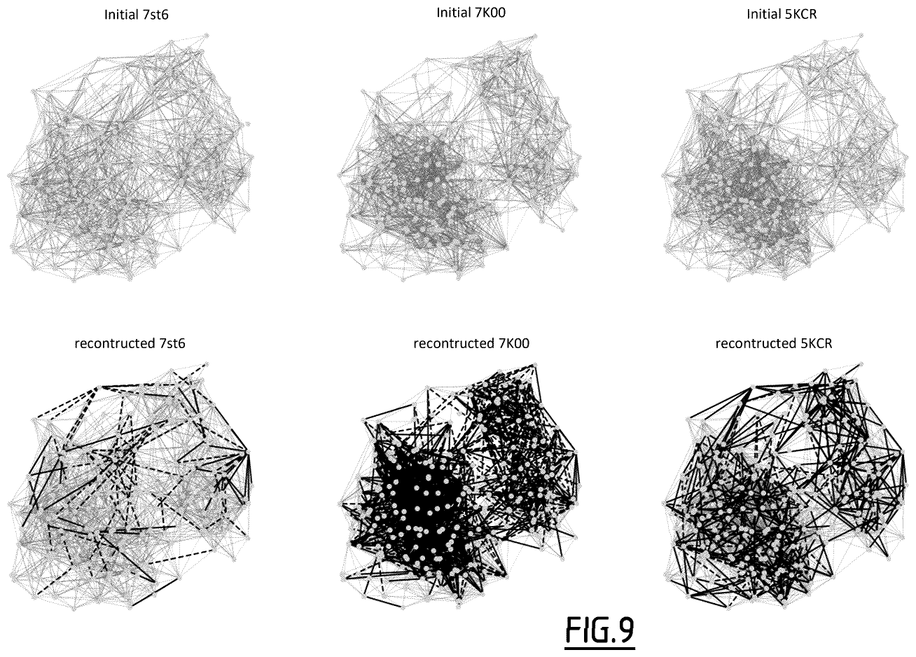

FIG. 8: illustrates a flow chart of an example method of calculating a sample large complex biomolecule based on a machine-learning (ML) model to validate whether a candidate drug compound is effective on the sample large complex biomolecule, according to aspects of the present invention disclosure. The example method may be performed by the computer system 610 of FIG. 6. The example method starts by identifying 810 a target set of compounds based on one or more of: a defined target clinical application, a set of desired characteristics, or a defined class of compounds. Each compound of the target set of compounds is pre-processed 820 to generate respective sets of feature data. The sets of feature data include a set of parameters based on a network analysis for the ML model to predict interaction of elements of the sample large complex biomolecule.

The sets of feature data are processed 830 with one or more trained machine learning models to produce predicted characteristic values for each compound of the target set of compounds for each of the set of desired characteristics. The one or more trained machine learning models are selected from a database of trained machine learning models based on at least the set of desired characteristics. A subset of the target set of compounds is identified 840 based on the predicted characteristic values.

In aspects, the sample large complex biomolecule includes at least one of a ribosome or ribozyme including RNA and proteins decomposable into at least one of secondary structures, tertiary structures, or domains defined in a use case. The target set of compounds includes at least one of antibiotics, anti-viral drugs, or anti-cancer drugs. The sample large complex biomolecule is of bacterial or eukaryote origin.

In aspects, the ML model includes a graphical neural network trained by operations including: receiving a training dataset of ribosomal structures; identifying one or more single elements for each of the training dataset of ribosomal structures; calculating, in the network analysis, interactions of the one or more single elements using a difference between a sum of SASAs of the one or more single elements independently and an actual SASA of the one or more single elements together; and recording a set of parameters based on the network analysis for the ML model to predict the interaction of the elements of the sample large complex biomolecule.

In aspects, the set of parameters includes at least one of: a shape parameter of a probability distribution of the interactions of the one or more single elements of the training dataset of ribosomal structures; a location parameter of the probability distribution of the interactions of the one or more single elements of the training dataset of ribosomal structures; a number of nodes each representing a single element of the training dataset of ribosomal structures, the number of nodes forming a network graph for the network analysis; a number of edges each linking two of the number of nodes; an average degree or connection of the number of nodes; a maximum degree or centrality of the number of nodes; a diameter of the network graph; an average path length of the network graph; a cluster coefficient of the network graph; a density of the network graph; an assortativity of the network graph; a neighborhood of one of the number of nodes. The one of the number of nodes has a maximum degree exceeding a threshold or a reference value; paths between two or more of the number of nodes having a respective maximum degree exceeding the threshold or the reference value; a number of residues associated with an output of the ML model, the output characterizing the interaction of elements of the sample large complex biomolecule.

### Example Aspects

Example 1. A computer-implemented method for calculating a sample large complex biomolecule based on a machine-learning (ML) model to validate whether a candidate drug compound is effective on the sample large complex biomolecule, the method comprising:
identifying a target set of compounds based on one or more of: a defined target clinical application, a set of desired characteristics, or a defined class of compounds;
pre-processing each compound of the target set of compounds to generate respective sets of feature data wherein the sets of feature data comprise a set of parameters based on a network analysis for the ML model to predict interaction of elements of the sample large complex biomolecule;
processing the sets of feature data with one or more trained machine learning models to produce predicted characteristic values for each compound of the target set of compounds for each of the set of desired characteristics, wherein the one or more trained machine learning models are selected from a database of trained machine learning models based on at least the set of desired characteristics; and
identifying a subset of the target set of compounds based on the predicted characteristic values.

Example 2. The method of Example 1, wherein:
the sample large complex biomolecule comprises at least one of a ribosome or ribozyme including RNA and proteins decomposable into at least one of secondary structures, tertiary structures, or domains defined in a use case;
the target set of compounds comprises at least one of antibiotics, anti-viral drugs, or anti-cancer drugs; and
the sample large complex biomolecule is of bacterial or eukaryote origin.

Example 3. The method of Example 1, wherein the ML model comprises a graphical neural network trained by operations comprising:
receiving a training dataset of ribosomal structures;
identifying one or more single elements for each of the training dataset of ribosomal structures;
calculating, in the network analysis, interactions of the one or more single elements using a difference between (1) a sum of surface accessible solvent areas (SASAs) of the one or more single elements independently and (2) an actual surface accessible solvent area (SASA) of the one or more single elements together; and recording a set of parameters based on the network analysis for the ML model to predict the interaction of the elements of the sample large complex biomolecule.

Example 4. The method of Example 3, wherein the set of parameters comprises at least one of:
a shape parameter of a probability distribution of the interactions of the one or more single elements of the training dataset of ribosomal structures;
a location parameter of the probability distribution of the interactions of the one or more single elements of the training dataset of ribosomal structures;
a number of nodes each representing a single element of the training dataset of ribosomal structures, the number of nodes forming a network graph for the network analysis;
a number of edges each linking two of the number of nodes;
an average degree or connection of the number of nodes;
a maximum degree or centrality of the number of nodes;
a diameter of the network graph;
an average path length of the network graph;
a cluster coefficient of the network graph;
a density of the network graph;
an assortativity of the network graph;
a neighborhood of one of the number of nodes, wherein the one of the number of nodes has a maximum degree exceeding a threshold or a reference value;
paths between two or more of the number of nodes having a respective maximum degree exceeding the threshold or the reference value;
a number of residues associated with an output of the ML model, the output characterizing the interaction of elements of the sample large complex biomolecule.

Example 5. The method of Example 3, wherein the SASAs and SASA are computed using Shrake Rupley method. Preferably, the radius of the solvent in said method should incorporate the b-factor of each node, using the pseudo solvent method described earlier.

Example 6. A method for calculating a sample large complex biomolecule, the method comprising:
identifying an action of a target set of compounds based on one or more of: a defined target clinical application, a set of desired characteristics, or a defined class of compounds;
pre-processing each compound of the target set of compounds to generate respective sets of feature data wherein the sets of feature data comprise a set of parameters based on a network analysis for a machine learning (ML) model to predict interaction of elements of the sample large complex biomolecule;
processing the sets of feature data with one or more trained machine learning models to produce predicted characteristic action for each compound of the target set of compounds for each of the set of desired characteristics, wherein the one or more trained machine learning models are selected from a database of trained machine learning models based on at least the set of desired characteristics; and
identifying a subset of the target set of compounds based on the predicted characteristic action.

Example 7. The method of Example 6, wherein:
the sample large complex biomolecule comprises at least one of a ribosome or ribozyme including RNA and proteins decomposable into at least one of secondary structures, tertiary structures, or domains defined in a use case;
the target set of compounds comprises at least one of antibiotics, anti-viral drugs, or anti-cancer drugs; and
the sample large complex biomolecule is of bacterial or eukaryote origin.

Example 8. The method of Example 7, wherein the ML model comprises a graphical neural network, and the method further comprising training the ML model by:
receiving a training dataset of ribosomal structures;
identifying one or more single elements for each of the training dataset of ribosomal structures;
calculating, in the network analysis, interactions of the one or more single elements using a difference between (1) a sum of surface accessible solvent areas (SASAs) of the one or more single elements independently and (2) an actual surface accessible solvent area (SASA) of the one or more single elements together; and
recording a set of parameters based on the network analysis for the ML model to predict the interaction of the elements of the sample large complex biomolecule.

Example 9. The method of Example 8, wherein the set of parameters comprises at least one of:
a shape parameter of a probability distribution of the interactions of the one or more single elements of the training dataset of ribosomal structures;
a location parameter of the probability distribution of the interactions of the one or more single elements of the training dataset of ribosomal structures;
a number of nodes each representing a single element of the training dataset of ribosomal structures, the number of nodes forming a network graph for the network analysis;
a number of edges each linking two of the number of nodes;
an average degree or connection of the number of nodes;
a maximum degree or centrality of the number of nodes;
a diameter of the network graph;
an average path length of the network graph;
a cluster coefficient of the network graph;
a density of the network graph;
an assortativity of the network graph;
a neighborhood of one of the number of nodes;
paths between two or more of the number of nodes having a respective maximum degree exceeding the threshold or the reference value; or
a number of residues associated with an output of the ML model, the output characterizing the interaction of elements of the sample large complex biomolecule.

Example 10. The method of Example 8, wherein the SASAs and SASA are computed using Shrake Rupley method. Preferably, the radius of the solvent in said method should incorporate the b-factor of each node, using the pseudo solvent method described earlier.

Example 11. A method for a computer system to calculate a sample ribosome structure based on a machine-learning (ML) model, the method comprising:
receiving an input of the sample ribosome structure for the ML model;
processing the input to generate an output that characterizes an interaction of elements of the sample ribosome structure; and
presenting, via a user-interface of the computer system, the output for validation or local targeting based on the interaction of the elements of the sample ribosome structure, wherein the ML model has been trained using a training dataset of ribosomal structures by:
   receiving the training dataset of ribosomal structures;
   identifying one or more single elements for each of the training dataset of ribosomal structures;
   calculating, in a network analysis, interactions of the one or more single elements using a difference between (1) a sum of surface accessible solvent areas (SASAs) of the one or more single elements independently and (2) an actual surface accessible solvent area (SASA) of the one or more single elements together; and
   recording a set of parameters based on the network analysis for the ML model to predict the interaction of the elements of the sample ribosome structure.

Example 12. The method of Example 11, wherein identifying the one or more single elements for each of the training dataset of ribosomal structures comprises:
separating each of the training dataset of ribosomal structures into constituent elements, including at least one of: 5S rRNAs, tRNAs, mRNAs, ribosomal proteins, 16S rRNAs, or 23S rRNAs; and
representing the constituent elements by a geometry having one or more perimeters.

Example 13. The method of Example 12, wherein the SASAs and SASA are computed using Shrake Rupley method. Preferably, the radius of the solvent in said method should incorporate the b-factor of each node, using the pseudo solvent method described earlier.

Example 14. The method of Example 12, further comprising:
comparing, in the network analysis, the training dataset of ribosomal structures using either an unsupervised clustering algorithm including at least one of: K-means clustering, principle component analysis, or a latent space of an autoencoder; or a supervised method such as a multilayer perceptron, a convolutional neural network, a transformer, or a recurrent neural network.

Example 15. The method of Example 11, wherein the ML model comprises a graphical neural network and wherein the set of parameters based on the network analysis for the ML model comprises at least one of:
a shape parameter of a probability distribution of the interactions of the one or more single elements of the training dataset of ribosomal structures;
a location parameter of the probability distribution of the interactions of the one or more single elements of the training dataset of ribosomal structures;
a number of nodes each representing a single element of the training dataset of ribosomal structures, the number of nodes forming a network graph for the network analysis;
a number of edges each linking two of the number of nodes;
an average degree or connection of the number of nodes;
a maximum degree or centrality of the number of nodes;
a diameter of the network graph;
an average path length of the network graph;
a cluster coefficient of the network graph;
a density of the network graph;
an assortativity of the network graph;
a neighborhood of one of the number of nodes;
paths between two or more of the number of nodes having a respective maximum degree exceeding the threshold or the reference value; or
a number of residues associated with an output of the ML model, the output characterizing the interaction of elements of the sample large complex biomolecule.

Example 16. The method of Example 15, wherein the output comprises at least one of:
a plurality of paths connecting elements of interests of the sample ribosome structure;
nodes that appear the most often in the plurality of paths;
residuals associated with the output characterizing the interaction of elements of the sample ribosome structure for local targeting; or
a subset of the plurality of paths susceptible to therapeutic targeting.

Example 17. The method of Example 15, wherein presenting the output comprises:
modeling an agent as one or more nodes of the network graph; and
determining the agent as active when at least one of the following conditions is satisfied:
   a number of edges has been changed exceeding a threshold;
   a path connecting predefined regions of interest of the ribosome has changed; or
   a difference between the sample ribosome structure and a reference ribosome structure exceeds a threshold value.

Example 18. A system for calculating a sample ribosome structure based on a machine-learning (ML) model, the system comprising:
a user-interface;
a memory; and
a processing device coupled to the memory, the processing device and the memory configured to:
   receive an input of the sample ribosome structure for the ML model;
   process the input to generate an output that characterizes an interaction of elements of the sample ribosome structure; and
   present, via the user-interface, the output for validation or local targeting based on the interaction of the elements of the sample ribosome structure, wherein the ML model has been trained using a training dataset of ribosomal structures by:
      receiving the training dataset of ribosomal structures;
      identifying one or more single elements for each of the training dataset of ribosomal structures;
      calculating, in a network analysis, interactions of the one or more single elements using a difference between (1) a sum of surface accessible solvent areas (SASAs) of the one or more single elements independently and (2) an actual surface accessible solvent area (SASA) of the one or more single elements together; and
      recording a set of parameters based on the network analysis for the ML model to predict the interaction of the elements of the sample ribosome structure.

Example 19. The system of Example 18, wherein the processing device and the memory are configured to identify the one or more single elements for each of the training dataset of ribosomal structures by:
separating each of the training dataset of ribosomal structures into constituent elements, including at least one of: 5S rRNAs, tRNAs, mRNAs, ribosomal proteins, 16S rRNAs, or 23S rRNAs; and
representing the constituent elements by a geometry having one or more perimeters.

Example 20. The system of Example 19, wherein the SASAs and SASA are computed using Shrake Rupley method. Preferably, the radius of the solvent in said method should incorporate the b-factor of each node, using the pseudo solvent method described earlier.

Example 21. The system of Example 19, wherein the processing device and the memory are further configured to:
compare, in the network analysis, the training dataset of ribosomal structures using either an unsupervised clustering algorithm including at least one of: K-means clustering, principle component analysis, or a latent space of an autoencoder; or a supervised method such as a multilayer perceptron, a convolutional neural network, a transformer, or a recurrent neural network.

Example 22. The system of Example 21, wherein the ML model comprises a graphical neural network and wherein the set of parameters based on the network analysis for the ML model comprises at least one of:
a shape parameter of a probability distribution of the interactions of the one or more single elements of the training dataset of ribosomal structures;
a location parameter of the probability distribution of the interactions of the one or more single elements of the training dataset of ribosomal structures;
a number of nodes each representing a single element of the training dataset of ribosomal structures, the number of nodes forming a network graph for the network analysis;
a number of edges each linking two of the number of nodes;
an average degree or connection of the number of nodes;
a maximum degree or centrality of the number of nodes;
a diameter of the network graph;
an average path length of the network graph;
a cluster coefficient of the network graph;
a density of the network graph;
an assortativity of the network graph;
a neighborhood of one of the number of nodes;
paths between two or more of the number of nodes having a respective maximum degree exceeding the threshold or the reference value; or
a number of residues associated with an output of the ML model, the output characterizing the interaction of elements of the sample large complex biomolecule.

Example 23. The system of Example 22, wherein the output comprises at least one of:
a plurality of paths connecting elements of interests of the sample ribosome structure;
nodes that appear the most often in the plurality of paths;
residuals associated with the output characterizing the interaction of elements of the sample ribosome structure for local targeting; or
a subset of the plurality of paths susceptible to therapeutic targeting.

Example 24. The system of Example 22, wherein presenting the output comprises:
modeling an agent as one or more nodes of the network graph; and
determining the agent as active when at least one of the following conditions is satisfied:
   a number of edges has been changed exceeding a threshold;
   a path connecting predefined regions of interest of the ribosome has changed; or
   a difference between the sample ribosome structure and a reference ribosome structure exceeds a threshold value.

Example 25. A non-transitory computer-readable storage medium including instructions that, when executed by a processing device to calculate a sample ribosome structure based on a machine-learning (ML) model, cause the processing device to:
receive an input of the sample ribosome structure for the ML model;
process the input to generate an output that characterizes an interaction of elements of the sample ribosome structure; and
present, via a user-interface, the output for validation or local targeting based on the interaction of the elements of the sample ribosome structure, wherein the ML model has been trained using a training dataset of ribosomal structures by:
   receiving the training dataset of ribosomal structures;
   identifying one or more single elements for each of the training dataset of ribosomal structures;
   calculating, in a network analysis, interactions of the one or more single elements using a difference between (1) a sum of surface accessible solvent areas (SASAs) of the one or more single elements independently and (2) an actual surface accessible solvent area (SASA) of the one or more single elements together; and
   recording a set of parameters based on the network analysis for the ML model to predict the interaction of the elements of the sample ribosome structure.

Example 26. The non-transitory computer-readable storage medium of Example 25, wherein the processing device is further to identify the one or more single elements for each of the training dataset of ribosomal structures by:
separating each of the training dataset of ribosomal structures into constituent elements, including at least one of: 5S rRNAs, tRNAs, mRNAs, ribosomal proteins, 16S rRNAs, or 23S rRNAs; and
representing the constituent elements by a geometry having one or more perimeters.

Example 27. The non-transitory computer-readable storage medium of Example 26, wherein the SASAs and SASA are computed using Shrake Rupley method. Preferably, the radius of the solvent in said method should incorporate the b-factor of each node, using the pseudo solvent method described earlier.

Example 28. The non-transitory computer-readable storage medium of Example 26, wherein the processing device is further to:
compare, in the network analysis, the training dataset of ribosomal structures using either an unsupervised clustering algorithm including at least one of: K-means clustering, principle component analysis, or a latent space of an autoencoder; or a supervised method such as a multilayer perceptron, a convolutional neural network, a transformer, or a recurrent neural network.

Example 29. The non-transitory computer-readable storage medium of Example 28, wherein the ML model comprises a graphical neural network and wherein the set of parameters based on the network analysis for the ML model comprises at least one of:
a shape parameter of a probability distribution of the interactions of the one or more single elements of the training dataset of ribosomal structures;
a location parameter of the probability distribution of the interactions of the one or more single elements of the training dataset of ribosomal structures;
a number of nodes each representing a single element of the training dataset of ribosomal structures, the number of nodes forming a network graph for the network analysis;
a number of edges each linking two of the number of nodes;
an average degree or connection of the number of nodes;
a maximum degree or centrality of the number of nodes;
a diameter of the network graph;
an average path length of the network graph;
a cluster coefficient of the network graph;
a density of the network graph;
an assortativity of the network graph;
a neighborhood of one of the number of nodes;
paths between two or more of the number of nodes having a respective maximum degree exceeding the threshold or the reference value; or
a number of residues associated with an output of the ML model, the output characterizing the interaction of elements of the sample large complex biomolecule.

Example 30. The non-transitory computer-readable storage medium of Example 29, wherein the output comprises at least one of:
a plurality of paths connecting elements of interests of the sample ribosome structure;
nodes that appear the most often in the plurality of paths;
residuals associated with the output characterizing the interaction of elements of the sample ribosome structure for local targeting; or
a subset of the plurality of paths susceptible to therapeutic targeting.

Example 31. The non-transitory computer-readable storage medium of Example 29, wherein the processing device is configured to present the output by:
modeling an agent as one or more nodes of the network graph; and
determining the agent as active when at least one of the following conditions is satisfied:
   a number of edges has been changed exceeding a threshold;
   a path connecting predefined regions of interest of the ribosome has changed; or
   a difference between the sample ribosome structure and a reference ribosome structure exceeds a threshold value.

What is described herein as example aspects can be combined with any feature and aspect of the description. Unless otherwise apparent from the context, all elements, steps or features described herein can be used in any combination with other elements, steps or features.

1. A method for identifying at least one biological characteristic of a complex molecular structure comprising:
S1) producing an interaction network representation of the complex molecular structure from a structural file of said complex molecular structure, wherein said representation comprises nodes connected by edges, each node representing either a molecule or a part of a molecule of the complex molecular structure, according to the following:
i. determining each functional structure of nucleic acid chains or protein as a respective node,
ii. for each node, calculating the node isolated solvent surface accessible area from the structural file using a pseudo probe solvent molecule,
iii. for each possible pair of nodes, calculating the pair's isolated solvent surface accessible area from the structural file using a pseudo probe solvent molecule,
iv. when the pair's isolated solvent surface accessible area is inferior to the sum of the isolated solvent surface accessible area of each node taken alone, then representing an edge connecting the two nodes of the pair,
S2) using the interaction network representation to identify at least one biological characteristic of the complex molecular structure.

2. The method according to claim 1, wherein in step ii) and iii), the b-factor of said node(s) in said structural file is taken into account to calculate the radius of the pseudo probe solvent molecule.

3. The method according to claim 2, wherein in step ii), the radius of a pseudo probe solvent molecule R_{probe} used to calculate the node isolated solvent surface accessible area is calculated as follows:
$${R}_{probe} ( {node}_{i} ) = {r}_{H 2 O} + {d}_{nodei}$$
wherein R_{probe}(nodeᵢ) is the radius of the pseudo probe for the nodeᵢ,
r_{H20} is the radius of water molecule,
and d_{nodei} is the mean displacement of the node with index i, and
wherein in step iii) the radius of a pseudo probe molecule R_{probe}(pairᵢⱼ) used to calculate the isolated solvent surface accessible area of the pair is calculated as follows:
$${R}_{probe} ( {pair}_{ij} ) = {r}_{H 2 O} + {d}_{nodei} + {d}_{nodej}$$
wherein R_{probe}(pairᵢⱼ) is the radius of the pseudo probe for the pair of nodes with indexes i and j,
r_{H20} is the radius of water molecule,
d_{nodei} is the mean displacement of the first node with index i of the pair, and
and d_{nodej} is the mean displacement of the second node with index j of the pair;
wherein the mean displacement is given by the relation:
$${d}_{n o d e i} = \sqrt{\frac{3 {B}_{i}}{8 {π}^{2}}}$$
wherein Bᵢ is the b-factor of the node with index i.

4. The method according to claim 1, wherein the step S2 comprises applying a mathematical transformation to the interaction network representation obtained in S1 to obtain a matrix, the matrix including rows of length N, where N is the total number of elements in the interaction network representation, each row representing a single element in the set of elements of the interaction network representation.

5. The method according to claim 1, wherein the identified biological characteristic of the complex molecular structure is a therapeutical target on said complex molecular structure, and wherein step S2) comprises:
a) finding paths connecting predefined regions of interest of the complex molecular structure and comprising less than 5 nodes, and
b) identifying as a therapeutical targets nodes from the paths found in step a), wherein paths are constituted of connected nodes and edges, each path comprising at least two nodes.

6. The method according to claim 1, wherein said method comprises:
a) producing several interaction network representations of a complex molecular structure in different configurations according to step S1),
b) training an autoencoder, wherein said autoencoder comprises an encoder, a decoder connected at output of the encoder, wherein the output of the encoder is a latent space, and wherein the output of the decoder is similar to the input of the encoder,
wherein interaction network representations of step a) are used as a training dataset of said autoencoder,
c) producing, as output of the trained autoencoder, simulated interaction network representations of said complex molecular structure, different simulated interaction network representations being produced by adapting respective parameters in the latent space;
the latent space preferably following a distribution among a probability distribution and a normal distribution with mean and standard deviation as parameters of the latent space.

7. The method according to claim 6, wherein the identified biological characteristic of the complex molecular structure is the region targeted by an active agent on said complex molecular structure, said method comprising:
a) producing several interaction network representations of a complex molecular structure in different configurations and simulated interaction network representations of said complex molecular structure,
b) producing an interaction network representation of said complex molecular structure with the active agent according to step S1),
c) determining a similarity between the interaction network representation of step b) and each interaction network representations of step a), from their output in the latent space, and selecting the network representation of step a) the most similar to the network representation of step b),
d) identifying the region of the complex molecular structure wherein paths have changed between the network representation of step c) and the network representation of step b), as the region of the complex molecular structure targeted by the active agent.

8. The method according to claim 6, wherein the identified biological characteristic of the complex molecular structure is the activity of an agent on said complex molecular structure, the method comprising:
a) producing several interaction network representations of a complex molecular structure in different configurations and simulated interaction network representations of said complex molecular structure,
b) producing an interaction network representation of said complex molecular structure with the agent according to step S1),
c) determining the similarity between the interaction network representation of step b) and each interaction network representations of step a), from their output in the latent space, and selecting the network representation of step a) the most similar to the network representation of step b),
d) identifying the agent as active on the complex molecular structure when at least one of the following conditions is verified between the interaction network representation of step b) and the one selected in step c):
i. the number of edges is significantly changed,
ii. a path connecting predefined regions of interest of the complex molecular structure is changed,
wherein each path is constituted of connected nodes and edges, each path comprising at least two nodes,
iii. the most similar network representation determined in step c) is a functional configuration of the complex molecular structure, and the similarity score between the interaction network representations of step c) and the interaction network representations of step b) is significantly low,
iv. the most similar network representation determined in step c) is a non-functional configuration of the complex molecular structure.

9. The method according to claim 1, wherein the identified biological characteristic is the similarity of two configurations of a complex molecular structure, wherein said method comprises:
a) producing several interaction network representations of said complex molecular structure in different configurations according to step S1,
b) training an autoencoder,
wherein said autoencoder comprises an encoder, a decoder connected at output of the encoder, wherein the output of the encoder is a latent space, and wherein the output of the decoder is similar to the input of the encoder,
wherein interaction network representations of step a) are used as a training dataset of said autoencoder,
c) using as input to the autoencoder interaction network representations of the two configurations to compare,
d) determining a similarity between the two configurations from their output in the latent space and/or from their output of the decoder.

10. The method according to claim 7, wherein the similarity is evaluated from a distance between two interaction network representations;

11. The method according to claim 10, wherein the distance between the two interaction network representations in the latent space is of the type chosen among the group comprising: a distance according to the Minkowski norm, mean square error and a cosine distance.

12. The method for identifying the specificity of an agent toward complex molecular structures comprising:
a) realizing the method according to claim 8, to identify the activity of the agent on a first complex molecular structure,
b) realizing the method according to claim 8, to identify the activity of the same agent on a second complex molecular structure,
and
c) when the agent is active on only one complex molecular structure, concluding that said agent is specific to said complex molecular structure.

13. The method according to claim 12, wherein the first and the second complex molecular structures are homologous but of different origin.

14. The method according to claim 1, wherein the complex molecular structure or complex molecular structures are ribosome(s).

15. A computer program comprising software instructions which, when executed by a computer, implement a method according to claim 1.

16. The method according to claim 4, wherein each column preferably representing a respective property of the interaction network representation.

17. The method according to claim 10, wherein the distance between two interaction network representations being preferably the distance between their output in the latent space and/or the distance between their output of the decoder.
